(19) United States
(12) Patent Application Publication
(10) Pub. No.: US 2014/0308332 A1
(43) Pub. Date: Oct. 16, 2014

(54) Compositions and Methods for Spine Fusion Procedures

(71) Applicants: Samuel E. Lynch, Franklin, TN (US); Leo B. Snel, Nashville, TN (US); Christopher K. Hee, Goleta, CA (US)

(72) Inventors: Samuel E. Lynch, Franklin, TN (US); Leo B. Snel, Nashville, TN (US); Christopher K. Hee, Goleta, CA (US)

(73) Assignee: BIOMIMETIC THERAPEUTICS, LLC, Franklin, TN (US)

(21) Appl. No.: 13/992,033

(22) PCT Filed: Dec. 13, 2011
(86) PCT No.: PCT/US11/64698

§ 371 Date: Sep. 26, 2013

Related U.S. Application Data
(60) Provisional application No. 61/422,649, filed on Dec. 13, 2010.

(51) Int. Cl. A61L 27/12; A61K 38/18

(52) U.S. Cl. CPC A61L 27/12; A61K 38/1858
USPC 424/423; 514/8.2

(57) ABSTRACT

The present invention provides compositions and methods for promoting fusion of bones in spine fusion procedures. In some embodiments, a method of performing a spine fusion procedure comprises providing a composition comprising PDGF disposed in a biocompatible matrix and applying the composition to a site of desired spine fusion.

### CROSS-REFERENCE TO RELATED APPLICATIONS

This application claims the benefit under 35 U.S.C. § 119(e) of U.S. Provisional Patent Application No. 61/442,649, filed Dec. 13, 2010, the entirety of which is hereby incorporated by reference.

### FIELD OF THE INVENTION

The present invention relates to compositions and methods useful for spine fusion procedures.

### BACKGROUND OF THE INVENTION

Spinal fusion is used in order to correct spinal deformities and for the treatment of fractured vertebrae, spinal instabilities, or chronic back pain. According to the American Academy of Orthopaedic Surgeons, more than 325,000 spinal fusions were performed in 2003, with approximately 162,000 of those in the lumbar spine (Spinal Fusion. Your Orthopaedic Connection September 2007 [cited 2009 January 20]; Available from: http://orthoinfo.aaos.org/topic.cfm?topic=A00348). One type of spine fusion procedure is an interbody fusion, in which all or part of the intervertebral disc is removed and a supporting spacer is inserted for support and to facilitate bone growth between the vertebral bodies. The bone growth is further enhanced with graft materials placed within the spacer. Autologous bone grafts, usually taken from the iliac crest, are commonly used to facilitate spinal fusion. Autograft is considered the “gold standard” due to its osteoconductive and osteoinductive properties, although there are limitations associated with its use including availability, donor site morbidity, pain, infection, nerve damage, and hemorrhage (Fowler, B. L., B. E. Dall, and D. E. Rowe, Complications associated with harvesting autogeneous iliac bone graft. American Journal of Orthopedics, 1995. 24: p. 895-903; Goulet, J., et al., Autogenous iliac crest bone graft: complications and functional assessment. Clinical Orthopedics and Related Research, 1997.339: p. 76-81; Vaccaro, A, The role of the osteoconductive scaffold in synthetic bone graft. Orthopedics, 2002. 25(5 Suppl): p. s571-s578). Allograft is an alternative to autograft that eliminates the complications associated with donor-site morbidity, however, the processing and sterilization of allograft can result in a reduction of biological activity compared to autograft (Khan, S. F., et al., The biology of bone grafting. Journal of the American Academy of Orthopaedic Surgeons, 2005. 13: p. 77-86).

In view of the difficulties associated with autologous and allograft bone grafts, it would be desirable to provide alternative osteogenic regeneration systems.

### BRIEF SUMMARY OF THE INVENTION

The present invention provides compositions and methods for use in spine fusion procedures. These compositions and methods promote fusion of spine bones. The present compositions and methods may facilitate the healing response in spine fusion procedures, for example, by facilitating bony union at fusion sites.

In one aspect of the invention is a method of promoting bone fusion in a spine fusion procedure, comprising administering to a site of desired spine fusion a composition comprising: a biocompatible matrix and a solution comprising platelet derived growth factor (PDGF), wherein the solution is incorporated in the biocompatible matrix, wherein the biocompatible matrix comprises a bone scaffolding material, and wherein the bone scaffolding material comprises a porous calcium phosphate or allograft. In some embodiments, the bone scaffolding material comprises calcium phosphate. In some embodiments, the calcium phosphate comprises β-tricalcium phosphate. In some embodiments, the bone scaffolding material comprises allograft. In some embodiments, the PDGF is present in the solution at a concentration from about 0.01 mg/ml to about 10.0 mg/ml. In some embodiments, the PDGF is present in the solution at a concentration from about 0.05 mg/ml to about 5.0 mg/ml. In some embodiments, the PDGF is present in the solution at a concentration from about 0.1 mg/ml to about 1.0 mg/ml. In some embodiments, the PDGF is present in the solution at a concentration from about 0.2 mg/ml to about 0.4 mg/ml. In some embodiments, the PDGF is present in the solution at a concentration of about 0.3 mg/ml. In some embodiments, the PDGF comprises PDGF-AA, PDGF-BB, PDGF-AB, PDGF-CC, PDGF-DD, or a mixture or a derivative thereof. In some embodiments, the PDGF comprises PDGF-BB. In some embodiments, the PDGF consists of PDGF-BB. In some embodiments, the PDGF-BB comprises at least 65% intact PDGF-BB homodimer. In some embodiments, the PDGF-BB is recombinant human (rh)PDGF-BB. In some embodiments, the solution comprises PDGF in a buffer. In some embodiments, the solution consists of PDGF in a buffer. In some embodiments, the buffer is sodium acetate. In some embodiments, the bone scaffolding material comprises particles in a range of about 50 microns to about 5000 microns in size. In some embodiments, the bone scaffolding material consists of particles in a range of about 50 microns to about 5000 microns in size. In some embodiments, the bone scaffolding material comprises particles in a range of about 100 microns to about 5000 microns in size. In some embodiments, the bone scaffolding material consists of particles in a range of about 100 microns to about 5000 microns in size. In some embodiments, the bone scaffolding material comprises particles in a range of about 100 microns to about 300 microns in size. In some embodiments, the bone scaffolding material consists of particles in a range of about 100 microns to about 300 microns in size. In some embodiments, the bone scaffolding material comprises particles in a range of about 1000 microns to about 2000 microns in size. In some embodiments, the bone scaffolding material consists of particles in a range of about 1000 microns to about 2000 microns in size. In some embodiments, the bone scaffolding material comprises particles in a range of about 250 microns to about 1000 microns in size. In some embodiments, the bone scaffolding material consists of particles in a range of about 250 microns to about 1000 microns in size. In some embodiments, the bone scaffolding material comprises particles in a range of about 1000 microns to about 3000 microns in size. In some embodiments, the bone scaffolding material consists of particles in a range of about 1000 microns to about 3000 microns in size. In some embodiments, the bone scaffolding material comprises porosity greater than about 25%. In some embodiments, the bone scaffolding material comprises porosity greater than about 40%. In some embodiments, the bone scaffolding material comprises porosity greater than about 50%. In some embodiments, the bone scaffolding material comprises porosity greater than about 80%. In some embodiments, the bone scaffolding material comprises porosity greater than about 90%. In some embodiments, the bone scaffolding material comprises macroporosity. In some embodiments, the bone scaffolding material has a porosity that facilitates cell migration into the matrix. In some embodiments, the bone scaffolding material comprises interconnected pores. In some embodiments, the bone scaffolding material is resorbable such that at least 80% of the bone scaffolding material is resorbed within one year of being implanted. In some embodiments, the solution is absorbed or adsorbed to the bone scaffolding material. In some embodiments, the bone scaffolding material is capable of absorbing an amount of the solution that is equal to at least about 25% of the bone scaffolding's own weight. In some embodiments, the bone scaffolding material is capable of absorbing an amount of the solution that is equal to at least about 50% of the bone scaffolding's own weight. In some embodiments, the bone scaffolding material is capable of absorbing an amount of the solution that is equal to at least about 100% of the bone scaffolding's own weight. In some embodiments, the bone scaffolding material is capable of absorbing an amount of the solution that is equal to at least about 200% of the bone scaffolding's own weight. In some embodiments, the bone scaffolding material is capable of absorbing an amount of the solution that is equal to at least about 300% of the bone scaffolding's own weight. In some embodiments, the biocompatible matrix further comprises a biocompatible binder. In some embodiments, the biocompatible binder comprises collagen. In some embodiments, the bone scaffolding material and collagen are present in a ratio of about 80:20. In some embodiments, the biocompatible matrix consists of calcium phosphate. In some embodiments, the biocompatible matrix consists of calcium phosphate and collagen. In some embodiments, the biocompatible matrix consists of allograft. In some embodiments, the biocompatible matrix consists of allograft and collagen. In some embodiments, the method comprises: performing a spine fusion procedure on a patient; applying the composition to a site of desired spine fusion; and, permitting bone fusion to occur at the site. In some embodiments, the spine fusion procedure is an interbody fusion procedure. In some embodiments, the spine fusion procedure is a lumbar fusion procedure. In some embodiments, the spine fusion procedure is a cervical fusion procedure. In some embodiments, the spine fusion procedure comprises accelerating bony union.

In another aspect, provided herein is the use of the compositions described herein in connection with the methods described herein, unless otherwise noted or as is clear from the specific context. The compositions described herein may also be used in the preparation of a medicament for use in the methods described herein.

In another aspect, the present invention provides a kit for use in a spine fusion procedure comprising a biocompatible matrix (or one or more components of a biocompatible matrix) in a first package and a solution comprising PDGF in a second package. The kit may further provide instructions for use in a method of performing a spine fusion procedure. In some embodiments, the solution comprises a predetermined concentration of PDGF. The concentration of the PDGF can be predetermined according to requirements of the spine fusion procedure(s) being performed. Moreover, in some embodiments, the biocompatible matrix can be present in the kit in a predetermined amount. In some embodiments, the biocompatible matrix in the kit comprises a bone scaffolding material, or a bone scaffolding material and a biocompatible binder. In some embodiments, the bone scaffolding material comprises a calcium phosphate, such as β-TCP. In some embodiments, the bone scaffolding material comprises allograft. In some embodiments, the binder comprises collagen. The amount of biocompatible matrix provided by a kit may relate to requirements of the spine fusion procedure(s) being performed. In some embodiments, the second package containing the PDGF solution comprises a vial. In some embodiments, the second package containing the PDGF solution comprises a syringe. A syringe can facilitate disposition of the PDGF solution in or on the biocompatible matrix for application at a surgical site, such as a site of bone fusion in a spine fusion procedure. In some embodiments, once the PDGF solution has been incorporated into the biocompatible matrix, the resulting composition is placed in a syringe and/or cannula for delivery to a site of desired spine fusion. Alternatively, the composition may be applied to the desired site with another application means, such as a surgical device, a spatula, spoon, knife, or equivalent device.

The present invention additionally provides methods for producing compositions for use in spine fusion procedures as well as methods of performing spine fusion procedures. In some embodiments, a method for producing a composition comprises providing a solution comprising PDGF, providing a biocompatible matrix, and disposing or incorporating the PDGF solution in the biocompatible matrix.

In another embodiment, a method of performing a spine fusion procedure comprises providing a composition comprising a PDGF solution disposed in a biocompatible matrix and applying the composition to a site of desired spine fusion. In some embodiments, a method of performing a spine fusion procedure comprises applying the composition to at least one site of desired bone fusion in a plurality of spinal bones. Applying the composition to a site of desired bone fusion, in some embodiments, comprises injecting the composition in the site of desired bone fusion.

In some embodiments, a method of performing a spine fusion procedure comprises surgically accessing a site of desired spine fusion, incorporating a composition comprising a PDGF solution disposed in a biocompatible matrix, applying the composition into the site of desired bone fusion, suturing soft tissues over the composition, and permitting cellular migration, ingrowth and infiltration into the composition for subsequent formation of bone.

In some embodiments, a spine fusion procedure comprises an interbody fusion procedure. In some embodiments, a spine fusion procedure comprises a posterolateral fusion procedure. In some embodiments, the spine fusion procedure is a lumbar fusion procedure. In some embodiments, the spine fusion procedure is a cervical fusion procedure. In some embodiments, the spine fusion procedure is a thoracic fusion procedure. In some embodiments, the spine fusion procedure is a sacral fusion procedure.

Accordingly, it is an object of the present invention to provide compositions comprising PDGF incorporated in a biocompatible matrix wherein the compositions are useful in facilitating the fusion of bones in spine fusion procedures.

Another object of the present invention is to provide spine fusion procedures using a composition comprising PDGF in a biocompatible matrix.

A further object of the present invention is to accelerate healing associated with bone fusion in spine fusion procedures.

These and other embodiments of the present invention are described in greater detail in the description which follows. These and other objects, features, and advantages of the present invention will become apparent after review of the following detailed description of the disclosed embodiments and claims.

### BRIEF DESCRIPTION OF THE DRAWINGS

FIGS. 1A and 1B show representative microCT images from each specimen grouped by treatment.

FIG. 2A and 2B show representative differential density analysis microCT images from freshly-prepared ABG, normal bone, freshly-prepared AIBG and specimens of the ABG-, AlBG- and Autograft-treated groups.

FIGS. 3A and 3B show representative histological images from each treatment group.

FIG. 4 shows representative histological images from ABG and AIBG treatment groups.

### DETAILED DESCRIPTION

All references cited herein, including without limitation, patents, patent applications and scientific references, are hereby incorporated herein by reference in their entirety.

The present invention provides compositions comprising a solution of PDGF incorporated in a biocompatible matrix, and methods for promoting the fusion of bone in spine fusion procedures. Spinal fusion, also known as spondylodesis or spondylosyndesis, is a surgical technique used to join two or more vertebrae. Types of spinal fusions include but are not limited to: interbody fusion, posterolateral fusion, and cervical diskectomy and fusion.

Interbody fusion places a bone graft (e.g. a composition of the invention) between the vertebrae in the area usually occupied by the intervertebral disc. In preparation for the spinal fusion, the disc may be removed entirely. A device may be placed between the vertebrae to maintain spine alignment and disc height. The intervertebral device may be, for example, a spacer. The intervertebral device may be made from, for example, plastic ortitanium. The fusion then occurs between the endplates of the vertebrae. Types of interbody fusion include: Anterior lumbar interbody fusion (ALIF), Posterior lumbar interbody fusion (PLIF), and Transforaminal lumbar interbody fusion (TLIF). In some embodiments, the fusion is augmented by a process called fixation, meaning the placement of metallic screws (pedicle screws often made from titanium), rods or plates, spacers, or cages to stabilize the vertebrae to facilitate bone fusion. During the fusion process, external bracing (orthotics) may be used.

Posterolateral fusion places the bone graft between the transverse processes in the back of the spine. These vertebrae may then be fixed in place with screws and/or wire through the pedicles of each vertebrae attaching to a metal rod on each side of the vertebrae.

### DEFINITIONS

As used herein, “promoting” or “facilitating” spinal fusion refers to a clinical intervention designed to desirably affect clinical progression of a spinal fusion procedure. Desirable effects of the clinical intervention include but are not limited to, for example, one or more of: increase in degree of bone density and/or acceleration of bone formation (e.g. acceleration of bone density) at the site of fusion, increase in degree of bony union or bone bridging and/or acceleration of bony union or bony bridging at the site of fusion, improvement in composition and/or structure of bone at bone fusion site (for example, closer resemblance to natural bone at the bone fusion site).

As used herein, the term “effective amount” refers to at least an amount effective, at dosages and for periods of time necessary, to achieve the desired therapeutic result. An effective amount can be provided in one or more administrations.

Reference to “about” a value or parameter herein also includes (and describes) embodiments that are directed to that value or parameter per se.

As used herein and in the appended claims, the singular forms “a,” “an,” and “the” include plural reference unless the context clearly indicates otherwise. For example, reference to a “PDGF homodimer” is a reference to one or multiple PDGF homodimers, and includes equivalents thereof known to those skilled in the art, and so forth.

It is understood that all aspects and embodiments of the invention described herein may include “comprising,” “consisting,” and “consisting essentially of” aspects and embodiments. It is to be understood that methods or compositions “consisting essentially of” the recited elements include only the specified steps or materials and those that do not materially affect the basic and novel characteristics of those methods and compositions.

“Bone scaffolding material” and “bone substituting agent” are used interchangeably herein.

### PDGF Solutions

In one aspect, a composition for spine fusion procedures provided by the present invention comprises a solution comprising PDGF and a biocompatible matrix, wherein the solution is disposed or incorporated in the biocompatible matrix. In some embodiments, PDGF is present in the solution in a concentration ranging from about 0.01 mg/ml to about 10 mg/ml, from about 0.05 mg/ml to about 5 mg/ml, or from about 0.1 mg/ml to about 1.0 mg/ml. PDGF may be present in the solution at any concentration within these stated ranges, including the upper limit and lower limit of each range. In other embodiments, PDGF is present in the solution at any one of the following concentrations: about 0.05 mg/ml; about 0.1 mg/ml; about 0.15 mg/ml; about 0.2 mg/ml; about 0.25 mg/ml; about 0.3 mg/ml; about 0.35 mg/ml; about 0.4 mg/ml; about 0.45 mg/ml; about 0.5 mg/ml; about 0.55 mg/ml; about 0.6 mg/ml; about 0.65 mg/ml; about 0.7 mg/ml; about 0.75 mg/ml; about 0.8 mg/ml; about 0.85 mg/ml; about 0.9 mg/ml; about 0.95 mg/ml; or about 1.0 mg/ml. It is to be understood that these concentrations are simply examples of particular embodiments, and that the concentration of PDGF may be within any of the concentration ranges stated above, including the upper limit and lower limit of each range.

Various amounts of PDGF may be used in the compositions of the present invention. Amounts of PDGF that are used, in some embodiments, include amounts in the following ranges: about 1 μg to about 50 mg, about 10 μg to about 25 mg, about 100 μg to about 10 mg, or about 250 μg to about 5 mg.

The concentration of PDGF or other growth factors in some embodiments of the present invention can be determined by using an enzyme-linked immunoassay as described in U.S. Pat. Nos. 6,221,625, 5,747,273, and 5,290,708, incorporated herein by reference, or any other assay known in the art for determining PDGF concentration. When provided herein, the molar concentration of PDGF is determined based on the molecular weight (MW) of PDGF dimer (e.g., PDGF-BB; MW about 25 kDa).

PDGF may comprise PDGF homodimers and/or heterodimers, including PDGF-AA, PDGF-BB, PDGF-AB, PDGF-CC, PDGF-DD, and mixtures and derivatives thereof. In some embodiments, PDGF comprises PDGF-BB. In another embodiment PDGF comprises a recombinant human (rh) PDGF, such as rhPDGF-BB.

PDGF, in some embodiments, can be obtained from natural sources. In other embodiments, PDGF can be produced by recombinant DNA techniques. In other embodiments, PDGF or fragments thereof may be produced using peptide synthesis techniques known to one of ordinary skill in the art, such as solid phase peptide synthetic. When obtained from natural sources, PDGF can be derived from biological fluids. Biological fluids, according to some embodiments, can comprise any treated or untreated fluid associated with living organisms including blood.

Biological fluids, in another embodiment, can also comprise blood components including platelet concentrate (PC), apheresed platelets, platelet-rich plasma (PRP), plasma, serum, fresh frozen plasma (FFP), and buffy coat (BC). Biological fluids, in a further embodiment, can comprise platelets separated from plasma and resuspended in a physiological fluid.

When PDGF is produced by recombinant DNA techniques, a DNA sequence encoding a single monomer (e.g., PDGF B-chain or A-chain), in some embodiments, can be inserted into cultured prokaryotic or eukaryotic cells for expression to subsequently produce the homodimer (e.g. PDGF-BB or PDGF-AA). In other embodiments, a PDGF heterodimer can be generated by inserting DNA sequences encoding for both monomeric units of the heterodimer into cultured prokaryotic or eukaryotic cells and allowing the translated monomeric units to be processed by the cells to produce the heterodimer (e.g. PDGF-AB). Commercially available GMP recombinant PDGF-BB can be obtained from Chiron Corporation (Emeryville, Calif.). Research grade rhPDGF-BB can be obtained from multiple sources including R&D Systems, Inc. (Minneapolis, Minn.), BD Biosciences (San Jose, Calif.), and Chemicon, International (Temecula, Calif.).

In some embodiments of the present invention, PDGF comprises PDGF fragments. In some embodiments rhPDGF-B comprises the following fragments: amino acid sequences 1-31, 1-32, 33-108, 33-109, and/or 1-108 of the entire B chain. The complete amino acid sequence (1-109) of the B chain of PDGF is provided in FIG. 15 of U.S. Pat. No. 5,516,896, the disclosure of which is hereby incorporated by reference in its entirety. It is to be understood that the rhPDGF-BB compositions of the present invention may comprise a combination of intact rhPDGF-B (1-109) and fragments thereof. Other fragments of PDGF may be employed such as those disclosed in U.S. Pat. No. 5,516,896. In accordance with one embodiment, the rhPDGF-BB comprises at least 65% of intact rhPDGF-B (1-109). In another embodiment, the rhPDGF-BB comprises at least 75%, 80%, 85%, 90%, 95%, or 99% of intact rhPDGF-B (1-109).

In some embodiments of the present invention, PDGF can be purified. Purified PDGF, as used herein, comprises compositions having greater than about 95% by weight PDGF prior to incorporation in solutions of the present invention. The solution may be any pharmaceutically acceptable solution. In other embodiments, the PDGF can be substantially purified. Substantially purified PDGF, as used herein, comprises compositions having about 5% to about 95% by weight PDGF prior to incorporation into solutions of the present invention. In some embodiments, substantially purified PDGF comprises compositions having about 65% to about 95% by weight PDGF prior to incorporation into solutions of the present invention. In other embodiments, substantially purified PDGF comprises compositions having about 70% to about 95%, about 75% to about 95%, about 80% to about 95%, about 85% to about 95%, or about 90% to about 95%, by weight PDGF, prior to incorporation into solutions of the present invention. Purified PDGF and substantially purified PDGF may be incorporated into scaffolds and binders.

In a further embodiment, PDGF can be partially purified. Partially purified PDGF, as used herein, comprises compositions having PDGF in the context of platelet rich plasma (PRP), fresh frozen plasma (FFP), or any other blood product that requires collection and separation to produce PDGF. Embodiments of the present invention contemplate that any of the PDGF isoforms provided herein, including homodimers and heterodimers, can be purified or partially purified. Compositions of the present invention containing PDGF mixtures may contain PDGF isoforms or PDGF fragments in partially purified proportions. Partially purified and purified PDGF, in some embodiments, can be prepared as described in U.S. patent application Ser. No. 11/159,533 (Publication No: 20060084602).

In some embodiments, solutions comprising PDGF are formed by solubilizing PDGF in aqueous media or in one or more buffers. Buffers suitable for use in PDGF solutions of the present invention can comprise, but are not limited to, carbonates, phosphates (e.g. phosphate buffered saline), histidine, acetates (e.g. sodium acetate), acidic buffers such as acetic acid and HCl, and organic buffers such as lysine, Tris buffers (e.g. tris(hydroxymethyl)aminoethane), N-2-hydroxyethylpiperazine-N′-2-ethanesulfonic acid (HEPES), and 3-(N-morpholino) propanesulfonic acid (MOPS). Buffers can be selected based on biocompatibility with PDGF and the buffer's ability to impede undesirable protein modification. Buffers can additionally be selected based on compatibility with host tissues. In some embodiments, sodium acetate buffer is used. The buffers can be employed at different molarities, for example, about 0.1 mM to about 100 mM, about 1 mM to about 50 mM, about 5 mM to about 40 mM, about 10 mM to about 30 mM, or about 15 mM to about 25 mM, or any molarity within these ranges. In some embodiments, an acetate buffer is employed at a molarity of about 20 mM.

In another embodiment, solutions comprising PDGF are formed by solubilizing lyophilized PDGF in water, wherein prior to solubilization the PDGF is lyophilized from an appropriate buffer.

Solutions comprising PDGF, according to embodiments of the present invention, can have a pH ranging from about 3.0 to about 8.0. In some embodiments, a solution comprising PDGF has a pH ranging from about 5.0 to about 8.0, from about 5.5 to about 7.0, or from about 5.5 to about 6.5, or any value within these ranges. The pH of solutions comprising PDGF, in same embodiments, can be compatible with the prolonged stability and efficacy of PDGF or any other desired biologically active agent. PDGF may be more stable in an acidic environment. Therefore, in accordance with one embodiment, the present invention comprises an acidic storage formulation of a PDGF solution. In accordance with this embodiment, the PDGF solution preferably has a pH from about 3.0 to about 7.0 or from about 4.0 to about 6.0. The biological activity of PDGF, however, can be optimized in a solution having a neutral pH range. Therefore, in a further embodiment, the present invention comprises a neutral pH formulation of a PDGF solution. In accordance with this embodiment, the PDGF solution has a pH from about 5.0 to about 8.0, from about 5.5 to about 7.0, or from about 5.5 to about 6.5. In accordance with a method of the present invention, an acidic PDGF solution is reformulated to a neutral pH composition. In accordance with a preferred embodiment of the present invention, the PDGF utilized in the solutions is rh-PDGF-BB. In a further embodiment, the pH of the PDGF containing solution can be altered to optimize the binding kinetics of PDGF to a biocompatible matrix.

The pH of solutions comprising PDGF, in some embodiments, can be controlled by the buffers recited herein. Various proteins demonstrate different pH ranges in which they are stable. Protein stabilities are primarily reflected by isoelectric points and charges on the proteins. The pH range can affect the conformational structure of a protein and the susceptibility of a protein to proteolytic degradation, hydrolysis, oxidation, and other processes that can result in modification to the structure and/or biological activity of the protein.

In some embodiments, solutions comprising PDGF can further comprise additional components, such as other biologically active agents. In other embodiments, solutions comprising PDGF can further comprise cell culture media, other stabilizing proteins such as albumin, antibacterial agents, protease inhibitors [e.g., ethylenediaminetetraacetic acid (EDTA), ethylene glycol-bis(beta-aminoethylether)-N, N,N′,N′-tetraacetic acid (EGTA), aprotinin, ε-aminocaproic acid (EACA), etc.] and/or other growth factors such as fibroblast growth factors (FGFs), epidermal growth factors (EGFs), transforming growth factors (TGFs), keratinocyte growth factors (KGFs), insulin-like growth factors (IGFs), bone morphogenetic proteins (BMPs), or other PDGFs including compositions of PDGF-AA, PDGF-BB, PDGF-AB, PDGF-CC and/or PDGF-DD.

### Biocompatible Matrix

The biocompatible matrix of the implant material is, or additionally includes, one or more bone substituting agents. The matrix may optionally further comprise a biocompatible binder.

### Bone Scaffolding Material

A biocompatible matrix, according to some embodiments of the present invention, comprises a bone scaffolding material. It is to be understood that the terms bone scaffolding material and bone substituting agent are used interchangeably in this patent application. The bone scaffolding material provides a framework or scaffold for new bone and tissue growth to occur. A bone substituting agent is one that can be used to permanently or temporarily replace bone. Following implantation, the bone substituting agent can be retained by the body or it can be resorbed by the body and replaced with bone. Exemplary bone substituting agents include, e.g., a calcium phosphate (e.g., tricalcium phosphate (e.g., β-TCP), hydroxyapatite, poorly crystalline hydroxyapatite, amorphous calcium phosphate, calcium metaphosphate, dicalcium phosphate dihydrate, heptacalcium phosphate, calcium pyrophosphate dihydrate, calcium pyrophosphate, and octacalcium phosphate), calcium sulfate, and allograft (e.g. mineralized bone, mineralized deproteinized xenograft, or demineralized bone (e.g., demineralized freeze-dried cortical or cancellous bone)). A bone scaffolding material, in some embodiments, comprises calcium phosphate. In some embodiments, calcium phosphate comprises β-TCP. In some embodiments, a bone scaffolding material comprises allograft. In some embodiments, biocompatible matrices may include calcium phosphate particles with or without biocompatible binders or bone allograft such as demineralized freeze dried bone allograft (DFDBA) or particulate demineralized bone matrix (DBM). In another embodiment, biocompatible matrices may include bone allograft such as DFDBA or DBM. In an embodiment, the carrier substance is bioresorbable. A bone scaffolding material, in some embodiments, comprises at least one calcium phosphate. In other embodiments, a bone scaffolding material comprises a plurality of calcium phosphates. Calcium phosphates suitable for use as a bone scaffolding material, in some embodiments of the present invention, have a calcium to phosphorus atomic ratio ranging from 0.5 to 2.0. In some embodiment, a biocompatible matrix comprises an allograft such as DFDBA or particulate DBM.

Non-limiting examples of calcium phosphates suitable for use as bone scaffolding materials comprise amorphous calcium phosphate, monocalcium phosphate monohydrate (MCPM), monocalcium phosphate anhydrous (MCPA), dicalcium phosphate dihydrate (DCPD), dicalcium phosphate anhydrous (DCPA), octacalcium phosphate (OCP), α-tricalcium phosphate, β-TCP, hydroxyapatite (OHAp), poorly crystalline hydroxapatite, tetracalcium phosphate (TTCP), heptacalcium decaphosphate, calcium metaphosphate, calcium pyrophosphate dihydrate, calcium pyrophosphate, carbonated calcium phosphate, or mixtures thereof.

In another embodiment, the bone substituting agent has a porous composition. Porosity is a desirable characteristic as it facilitates cell migration and infiltration into the implant material so that the infiltrating cells can secrete extracellular bone matrix. Porosity also provides access for vascularization. Porosity also provides a high surface area for enhanced resorption and release of active substances, as well as increased cell-matrix interaction. The composition can be provided in a shape suitable for implantation (e.g., a sphere, a cylinder, or a block) or it can be sized and shaped prior to use. In a preferred embodiment, the bone substituting agent is a calcium phosphate (e.g., β-TCP). Porous bone scaffolding materials, according to some embodiments, can comprise pores having diameters ranging from about 1 μm to about 1 mm. In some embodiments, a bone scaffolding material comprises macropores having diameters ranging from about 100 μm to about 1 mm. In another embodiment, a bone scaffolding material comprises mesopores having diameters ranging from about 10 μm to about 100 μm. In a further embodiment, a bone scaffolding material comprises micropores having diameters less than about 10 μm. Embodiments of the present invention contemplate bone scaffolding materials comprising macropores, mesopores, and micropores or any combination thereof. In some embodiments, the bone scaffolding material comprises interconnected pores. In some embodiments, the bone scaffolding material comprises non-interconnected pores. In some embodiments, the bone scaffolding material comprises interconnected and non-interconnected pores.

A porous bone scaffolding material, in some embodiments, has a porosity greater than about 25% or greater than about 40%. In another embodiment, a porous bone scaffolding material has a porosity greater than about 50%, greater than about 60%, greater than about 65%, greater than about 70%, greater than about 80%, or greater than about 85%. In a further embodiment, a porous bone scaffolding material has a porosity greater than about 90%. In some embodiments, a porous bone scaffolding material comprises a porosity that facilitates cell migration into the scaffolding material.

In some embodiments, a bone scaffolding material comprises a plurality of particles. A bone scaffolding material, for example, can comprise a plurality of calcium phosphate particles. Particles of a bone scaffolding material, in some embodiments, can individually demonstrate any of the pore diameters and porosities provided herein for the bone scaffolding. In other embodiments, particles of a bone scaffolding material can form an association to produce a matrix having any of the pore diameters or porosities provided herein for the bone scaffolding material.

Bone scaffolding particles may be mm, μm or submicron (nm) in size. Bone scaffolding particles, in some embodiments, have an average diameter ranging from about 1 μm to about 5 mm. In other embodiments, particles have an average diameter ranging from about 1 mm to about 2 mm, from about 1 mm to about 3 mm, or from about 250 μm to about 750 μm. Bone scaffolding particles, in another embodiment, have an average diameter ranging from about 100 μm to about 300 μm. In a further embodiment, the particles have an average diameter ranging from about 75 μm to about 300 μm. In additional embodiments, bone scaffolding particles have an average diameter less than about 25 μm, less than about 1 μm and, in some cases, less than about 1 mm. In some embodiments, a bone scaffolding particles have an average diameter ranging from about 100 μm to about 5 mm or from about 100 μm to about 3 mm. In other embodiments, bone scaffolding particles have an average diameter ranging from about 250 μm to about 2 mm, from about 250 μm to about 1 mm, from about 200 μm to about 3 mm. Particles may also be in the range of about 1 nm to about 1000 nm, less than about 500 nm or less than about 250 nm.

Bone scaffolding particles, in some embodiments, have a diameter ranging from about 1 μm to about 5 mm. In other embodiments, particles have a diameter ranging from about 1 mm to about 2 mm, from about 1 mm to about 3 mm, or from about 250 μm to about 750 μm. Bone scaffolding particles, in another embodiment, have a diameter ranging from about 100 μm to about 300 μm. In a further embodiment, the particles have a diameter ranging from about 75 μm to about 300 μm. In additional embodiments, bone scaffolding particles have a diameter less than about 25 μm, less than about 1 μm and, in some cases, less than about 1 mm. In some embodiments, a bone scaffolding particles have a diameter ranging from about 100 μm to about 5 mm or from about 100 μm to about 3 mm. In other embodiments, bone scaffolding particles have a diameter ranging from about 250 μm to about 2 mm, from about 250 μm to about 1 mm, from about 200 μm to about 3 mm. Particles may also be in the range of about 1 nm to about 1000 nm, less than about 500 nm or less than about 250 nm.

Bone scaffolding materials, according to some embodiments, can be provided in a shape suitable for implantation (e.g., a sphere, a cylinder, or a block). In other embodiments, bone scaffolding materials are moldable, extrudable, and/or injectable. Moldable, extrudable, and/or injectable bone scaffolding materials can facilitate efficient placement of compositions of the present invention in and around target sites in bone and between bones at sites of desired bone fusion during spine fusion procedures. In some embodiments, moldable bone scaffolding materials can be applied to sites of bone fusion with a spatula or equivalent device. In some embodiments, bone scaffolding materials are flowable. Flowable bone scaffolding materials, in some embodiments, can be applied to sites of bone fusion through a syringe and needle or cannula. In some embodiments, bone scaffolding materials harden in vivo.

In some embodiments, bone scaffolding materials are bioresorbable. A bone scaffolding material, in some embodiments, can be at least 30%, 40%, 50%, 60%, 70%, 75% or 90% resorbed within one year subsequent to in vivo implantation. In another embodiment, a bone scaffolding material can be resorbed at least 5%, 10%, 20%, 30%, 40%, 50%, 60%, 70%, 75% or 90% within 1, 3, 6, 9, 12, or 18 months of in vivo implantation. Bioresorbability will be dependent on: (1) the nature of the matrix material (i.e., its chemical make up, physical structure and size); (2) the location within the body in which the matrix is placed; (3) the amount of matrix material that is used; (4) the metabolic state of the patient (diabetic/non-diabetic, osteoporotic, smoker, old age, steroid use, etc.); (5) the extent and/or type of injury treated; and (6) the use of other materials in addition to the matrix such as other bone anabolic, catabolic and anti-catabolic factors.

### Bone Scaffolding Material and Biocompatible Binder

In another embodiment, a biocompatible matrix comprises a bone scaffolding material and a biocompatible binder. Bone scaffolding materials in some embodiments of a biocompatible matrix further comprising a biocompatible binder are consistent with those provided hereinabove.

Biocompatible binders, according to some embodiments, can comprise materials operable to promote cohesion between combined substances. A biocompatible binder, for example, can promote adhesion between particles of a bone scaffolding material in the formation of a biocompatible matrix. In certain some embodiments, the same material may serve as both a scaffolding material and a binder if such material acts to promote cohesion between the combined substances and provides a framework for new bone growth to occur.

Biocompatible binders, in some embodiments, can comprise collagen, polysaccharides, nucleic acids, carbohydrates, proteins, polypeptides, synthetic polymers, poly(α-hydroxy acids), poly(lactones), poly(amino acids), poly(anhydrides), polyurethanes, poly(orthoesters), poly(anhydride-co-imides), poly(orthocarbonates), poly(α-hydroxy alkanoates), poly(dioxanones), poly(phosphoesters), polylactic acid, poly(L-lactide) (PLLA), poly(D,L-lactide) (PDLLA), polyglycolide (PGA), poly(lactide-co-glycolide (PLGA), poly(L-lactide-co-D,L-lactide), poly(D,L-lactide-co-trimethylene carbonate), polyglycolic acid, polyhydroxybutyrate (PHB), poly(ε-caprolactone), poly(δ-valerolactone), poly(γ-butyrolactone), poly(caprolactone), polyacrylic acid, polycarboxylic acid, poly(allylamine hydrochloride), poly(diallyldimethylammonium chloride), poly(ethyleneimine), polypropylene fumarate, polyvinyl alcohol, polyvinylpyrrolidone, polyethylene, polymethylmethacrylate, carbon fibers, poly(ethylene glycol), poly(ethylene oxide), poly(vinyl alcohol), poly(vinylpyrrolidone), poly(ethyloxazoline), poly(ethylene oxide)-co-poly(propylene oxide) block copolymers, poly(ethylene terephthalate)polyamide, and copolymers and mixtures thereof.

Biocompatible binders, in other embodiments, can comprise alginic acid, arabic gum, guar gum, xantham gum, gelatin, chitin, chitosan, chitosan acetate, chitosan lactate, chondroitin sulfate, lecithin, N,O-carboxymethyl chitosan, phosphatidylcholine derivatives, a dextran (e.g., α-cyclodextrin, β-cyclodextrin, γ-cyclodextrin, or sodium dextran sulfate), fibrin glue, lecithin, glycerol, hyaluronic acid, sodium hyaluronate, a cellulose (e.g., methylcellulose, carboxymethylcellulose, hydroxypropyl methylcellulose, or hydroxyethyl cellulose), a glucosamine, a proteoglycan, a starch (e.g., hydroxyethyl starch or starch soluble), lactic acid, a pluronic acid, sodium glycerophosphate, glycogen, a keratin, silk, and derivatives and mixtures thereof.

In some embodiments, the binder comprises collagen. In some embodiments, the collagen comprises Type I collagen. In some embodiments, the collagen comprises bovine Type I collagen. In some embodiments, a biocompatible binder comprises hyaluronic acid.

In some embodiments, a biocompatible binder is water-soluble. A water-soluble binder can dissolve from the biocompatible matrix shortly after its implantation, thereby introducing macroporosity into the biocompatible matrix. Macroporosity, as discussed herein, can increase the osteoconductivity of the implant material by enhancing the access and, consequently, the remodeling activity of the osteoclasts and osteoblasts at the implant site.

In some embodiments, a biocompatible binder can be present in a biocompatible matrix in an amount ranging from about 1 weight percent to about 70 weight percent, about 5 weight percent to about 50 weight percent, about 10 weight percent to about 40 weight percent, about 15 weight percent to about 35 weight percent, or about 15 weight percent to about 25 weight percent of the biocompatible matrix. In a further embodiment, a biocompatible binder can be present in an amount of about 20 weight percent of the biocompatible matrix.

A biocompatible matrix comprising a bone scaffolding material and a biocompatible binder, according to some embodiments, can be flowable, moldable, and/or extrudable. In such embodiments, a biocompatible matrix can be in the form of a paste or putty. A biocompatible matrix in the form of a paste or putty, in some embodiments, can comprise particles of a bone scaffolding material adhered to one another by a biocompatible binder.

A biocompatible matrix in paste or putty form can be molded into the desired implant shape or can be molded to the contours of the implantation site. In some embodiments, a biocompatible matrix in paste or putty form can be injected into an implantation site with a syringe or cannula.

In some embodiments, a biocompatible matrix in paste or putty form does not harden and retains a flowable and moldable form subsequent to implantation. In other embodiments, a paste or putty can harden subsequent to implantation, thereby reducing matrix flowability and moldability.

A biocompatible matrix comprising a bone scaffolding material and a biocompatible binder, in some embodiments, can also be provided in a predetermined shape including a block, sphere, or cylinder or any desired shape, for example a shape defined by a mold or a site of application.

A biocompatible matrix comprising a bone scaffolding material and a biocompatible binder, in some embodiments, is bioresorbable as described above. A biocompatible matrix, in such embodiments, can be resorbed within one year of in vivo implantation. In another embodiment, a biocompatible matrix comprising a bone scaffolding material and a biocompatible binder can be resorbed within 1, 3, 6, or 9 months of in vivo implantation. Bioresorbablity will be dependent on: (1) the nature of the matrix material (i.e., its chemical make up, physical structure and size); (2) the location within the body in which the matrix is placed; (3) the amount of matrix material that is used; (4) the metabolic state of the patient (diabetic/non-diabetic, osteoporotic, smoker, old age, steroid use, etc.); (5) the extent and/or type of injury treated; and (6) the use of other materials in addition to the matrix such as other bone anabolic, catabolic and anti-catabolic factors.

While the following describes particular embodiments with reference to a bone scaffolding material comprising β-TCP and/or a biocompatible binder comprising collagen, it is to be understood that other embodiments of the invention may be produced by substituting other bone scaffolding material(s) (e.g. another calcium phosphate, calcium sulfate, or allograft) for the β-TCP, and/or by substituting other binder(s) for the collagen.

Bone Scaffolding Comprising β-Tricalcium Phosphate

In some embodiments, a bone scaffolding material for use as a biocompatible matrix can comprise β-TCP. β-TCP, according to some embodiments, can comprise a porous structure having multidirectional and interconnected pores of varying diameters. In some embodiments, β-TCP comprises a plurality of pockets and non-interconnected pores of various diameters in addition to the interconnected pores. The porous structure of β-TCP, in some embodiments, comprises macropores having diameters ranging from about 100 μm to about 1 mm, mesopores having diameters ranging from about 10 μm to about 100 μm, and micropores having diameters less than about 10 μm. Macropores and micropores of the β-TCP can facilitate osteoinduction and osteoconduction while macropores, mesopores and micropores can permit fluid communication and nutrient transport to support bone regrowth throughout the β-TCP biocompatible matrix.

In comprising a porous structure, β-TCP, in some embodiments, can have a porosity greater than 25% or greater than about 40%. In other embodiments, β-TCP can have a porosity greater than 50%, greater than about 60%, greater than about 65%, greater than about 70%, greater than about 75%, greater than about 80%, or greater than about 85%. In a further embodiment, β-TCP can have a porosity greater than about 90%. In some embodiments, (β-TCP can have a porosity that facilitates cell migration into the β-TCP.

In some embodiments, a bone scaffolding material comprises β-TCP particles. β-TCP particles, in some embodiments, can individually demonstrate any of the pore diameters and porosities provided herein for β-TCP. In other embodiments, β-TCP particles of a bone scaffolding material can form an association to produce a matrix having any of the pore diameters or porosities provided herein for the bone scaffolding material. Porosity may facilitate cell migration and infiltration into the matrix for subsequent bone formation. β-TCP particles, in some embodiments, have an average diameter ranging from about 1 μm to about 5 mm. In other embodiments, β-TCP particles have an average diameter ranging from about 1 mm to about 2 mm, from about 1 mm to about 3 mm, from about 250 μm to about 750 μm, from about 250 μm to about 1 mm, from about 250 μm to about 2 mm, or from about 200 μm to about 3 mm. In another embodiment, β-TCP particles have an average diameter ranging from about 100 μm to about 300 μm. In a further embodiment, β-TCP particles have an average diameter ranging from about 75 μm to about 300 μm. In additional embodiments, β-TCP particles have an average diameter less than about 25 μm, average diameter less than about 1 μm, or less than about 1 mm. In some embodiments, p-TCP particles have an average diameter ranging from about 100 μm to about 5 mm or from about 100 μm to about 3 mm.

A biocompatible matrix comprising β-TCP particles, in some embodiments, can be provided in a shape suitable for implantation (e.g., a sphere, a cylinder, or a block). In other embodiments, a β-TCP bone scaffolding material can be moldable, extrudable, and/or injectable thereby facilitating placement of the matrix in and around target sites of desired bone fusion during spine fusion procedures. Flowable matrices may be applied through syringes, tubes, or spatulas or equivalent devices. Flowable β-TCP bone scaffolding materials, in some embodiments, can be applied to sites of bone fusion through a syringe and needle or cannula. In some embodiments, β-TCP bone scaffolding materials harden in vivo.

A β-TCP bone scaffolding material, according to some embodiments, is bioresorbable. In some embodiments, a β-TCP bone scaffolding material can be at least 30%, 40%, 50%, 60%, 65%, 70%, 75%, 80%, or 85% resorbed one year subsequent to in vivo implantation. In another embodiment, a β-TCP bone scaffolding material can be greater than about 90% resorbed one year subsequent to in vivo implantation.

### Biocompatible Matrix Comprising β-TCP and Collagen

In some embodiments, a biocompatible matrix can comprise a β-TCP bone scaffolding material and a biocompatible collagen binder. β-TCP bone scaffolding materials suitable for combination with a collagen binder are consistent with those provided hereinabove.

A collagen binder, in some embodiments, can comprise any type of collagen, including Type I, Type II, and Type III collagens. In some embodiments, a collagen binder comprises a mixture of collagens, such as a mixture of Type I and Type II collagen. In other embodiments, a collagen binder is soluble under physiological conditions. Other types of collagen present in bone or musculoskeletal tissues may be employed. Recombinant, synthetic and naturally occurring forms of collagen may be used in the present invention.

A biocompatible matrix, according to some embodiments, can comprise a plurality of β-TCP particles adhered to one another with a collagen binder. β-TCP particles suitable for use with a collagen binder can comprise any of the β-TCP particles described herein. In some embodiments, β-TCP particles suitable for combination with a collagen binder have an average diameter ranging from about 1 μm to about 5 mm. In another embodiment, β-TCP particles suitable for combination with a collagen binder have an average diameter ranging from about 1 μm to about 1 mm, from about 1 mm to about 2 mm, from about 1 mm to about 3 mm, from about 250 μm to about 750 μm, from about 250 μm to about 1 mm, from about 250 μm to about 2 mm, from about 200 μm to about 1 mm, or from about 200 μm to about 3 mm. β-TCP particles, in other embodiments, have an average diameter ranging from about 100 μm to about 300 μm. In a further embodiment, p-TCP particles suitable for combination with a collagen binder have an average diameter ranging from about 75 μm to about 300 μm. In additional embodiments, β-TCP particles suitable for combination with a collagen binder have an average diameter less than about 25 μm and, less than about 1 mm or less than about 1 μm. In some embodiments, β-TCP particles suitable for combination with a collagen binder have an average diameter ranging from about 100 μm to about 5 mm or from about 100 μm to about 3 mm. β-TCP particles, in some embodiments, can be adhered to one another by the collagen binder so as to produce a biocompatible matrix having a porous structure. In some embodiments, a biocompatible matrix comprising β-TCP particles and a collagen binder can comprise pores having diameters ranging from about 1 μm to about 1 mm. A biocompatible matrix comprising β-TCP particles and a collagen binder can comprise macropores having diameters ranging from about 100 μm to about 1 mm, mesopores having diameters ranging from about 10 μm to 100 μm, and micropores having diameters less than about 10 μm.

A biocompatible matrix comprising β-TCP particles and a collagen binder can have a porosity greater than about 25% or greater than 40%. In another embodiment, the biocompatible matrix can have a porosity greater than about 50%, greater than about 60%, greater than about 65%, greater than about 70%, greater than about 80%, or greater than about 85%. In a further embodiment, the biocompatible matrix can have a porosity greater than about 90%. Porosity facilitates cell migration and infiltration into the matrix for subsequent bone formation.

A biocompatible matrix comprising β-TCP particles, in some embodiments, can comprise a collagen binder in an amount ranging from about 1 weight percent to about 70 weight percent, from about 5 weight percent to about 50 weight percent, from about 10 weight percent to about 40 weight percent, from about 15 weight percent to about 35 weight percent, or from about 15 weight percent to about 25 weight percent of the biocompatible matrix. In a further embodiment, a collagen binder can be present in an amount of about 20 weight percent of the biocompatible matrix.

A biocompatible matrix comprising β-TCP particles and a collagen binder, according to some embodiments, can be flowable, moldable, and/or extrudable. In such embodiments, the biocompatible matrix can be in the form of a paste or putty. A paste or putty can be molded into the desired implant shape or can be molded to the contours of the implantation site. In some embodiments, a biocompatible matrix in paste or putty form comprising β-TCP particles and a collagen binder can be injected into an implantation site with a syringe or cannula.

In some embodiments, a biocompatible matrix in paste or putty form comprising β-TCP particles and a collagen binder can retain a flowable and moldable form when implanted. In other embodiments, the paste or putty can harden subsequent to implantation, thereby reducing matrix flowability and moldability.

A biocompatible matrix comprising β-TCP particles and a collagen binder, in some embodiments, can be provided in a predetermined shape such as a block, sphere, or cylinder.

A biocompatible matrix comprising β-TCP particles and a collagen binder can be resorbable. In some embodiments, a biocompatible matrix comprising β-TCP particles and a collagen binder can be at least 30%, 40%, 50%, 60%, 70%, 75%, or 90% resorbed one year subsequent to in vivo implantation. In another embodiment, this matrix can be resorbed at least 5%, 10%, 20%, 30%, 40%, 50%, 60%, 70%, 75% or 90% within 1, 3, 6, 9, 12, or 18 months subsequent to in vivo implantation.

A solution comprising PDGF can be disposed in a biocompatible matrix to produce a composition for promoting bone fusion in spine fusion procedures according to embodiments of the present invention.

### Incorporating PDGF Solution in a Biocompatible Matrix

The present invention provides methods for producing compositions for use in spine fusion procedures. In some embodiments, a method for producing a composition for promoting the fusion of bone comprises providing a solution comprising PDGF, providing a biocompatible matrix, and incorporating the solution in the biocompatible matrix. PDGF solutions and biocompatible matrices suitable for combination are consistent with those described hereinabove.

In some embodiments, a PDGF solution can be incorporated in a biocompatible matrix by soaking the biocompatible matrix in the PDGF solution. A PDGF solution, in another embodiment, can be incorporated in a biocompatible matrix by injecting the biocompatible matrix with the PDGF solution. In some embodiments, injecting a PDGF solution can comprise incorporating the PDGF solution in a syringe and expelling the PDGF solution into the biocompatible matrix to saturate the biocompatible matrix.

The biocompatible matrix, according to some embodiments, can be in a predetermined shape, such as a brick or cylinder, prior to receiving a PDGF solution. Subsequent to receiving a PDGF solution, the biocompatible matrix can have a paste or putty form that is flowable, extrudable, and/or injectable. In other embodiments, the biocompatible matrix can already demonstrate a flowable paste or putty form prior to receiving a solution comprising PDGF.

### Compositions Further Comprising Biologically Active Agents

The compositions described herein for promoting and/or facilitating bone fusion in spine fusion procedures, according to some embodiments, can further comprise one or more biologically active agents in addition to PDGF. Biologically active agents that can be incorporated into compositions of the present invention in addition to PDGF can comprise organic molecules, inorganic materials, proteins, peptides, nucleic acids (e.g., genes, gene fragments, small insert ribonucleic acids [si-RNAs], gene regulatory sequences, nuclear transcriptional factors, and antisense molecules), nucleoproteins, polysaccharides (e.g., heparin), glycoproteins, and lipoproteins. Non-limiting examples of biologically active compounds that can be incorporated into compositions of the present invention, including, e.g., anti-cancer agents, antibiotics, analgesics, anti-inflammatory agents, immunosuppressants, enzyme inhibitors, antihistamines, hormones, muscle relaxants, prostaglandins, trophic factors, osteoinductive proteins, growth factors, and vaccines, are disclosed in U.S. patent application Ser. No. 11/159,533 (Publication No: 20060084602). In some embodiments, biologically active compounds that can be incorporated into compositions of the present invention include osteoinductive factors such as insulin-like growth factors, fibroblast growth factors, or other PDGFs. In accordance with other embodiments, biologically active compounds that can be incorporated into compositions of the present invention preferably include osteoinductive and osteostimulatory factors such as bone morphogenetic proteins (BMPs), BMP mimetics, calcitonin, calcitonin mimetics, statins, statin derivatives, or parathyroid hormone. Preferred factors also include protease inhibitors, as well as osteoporotic treatments that decrease bone resorption including bisphosphonates, and antibodies to receptor activator of NF-kB ligand (RANK) ligand.

Standard protocols and regimens for delivery of additional biologically active agents are known in the art. Additional biologically active agents can be introduced into compositions of the present invention in amounts that allow delivery of an appropriate dosage of the agent to the implant site. In most cases, dosages are determined using guidelines known to practitioners and applicable to the particular agent in question. The amount of an additional biologically active agent to be included in a composition of the present invention can depend on such variables as the type and extent of the condition, the overall health status of the particular patient, the formulation of the biologically active agent, release kinetics, and the bioresorbability of the biocompatible matrix. Standard clinical trials may be used to optimize the dose and dosing frequency for any particular additional biologically active agent.

A composition for promoting bone fusion in spine fusion procedures, according to some embodiments, can further comprise the addition of other bone grafting materials with PDGF including autologous bone marrow, autologous platelet extracts, and synthetic bone matrix materials.

### Methods of Performing Spine Fusion Procedures

The present invention also provides methods of performing spine fusion procedures. In some embodiments, a method of performing a spine fusion procedure comprises providing a composition comprising a PDGF solution incorporated in a biocompatible matrix and applying the composition to a site of desired spine fusion. A composition comprising a PDGF solution incorporated in a biocompatible matrix, for example, can be packed into a site of desired spine fusion. In some embodiments, the composition can be packed such that the composition is in contact with the entire surface area of the bones in the bone fusion site. The composition may additionally be applied to the vicinity of the bone fusion site to further strengthen the fused bones.

Vertebral bones in any portion of the spine may be fused using the compositions and methods of the present invention, including the cervical, thoracic, lumbar, and sacral regions.

In another embodiment, a method of the present invention comprises accelerating bony union in a spine fusion procedure wherein accelerating bony union comprises providing a composition comprising a PDGF solution disposed in a biocompatible matrix and applying the composition to at least one site of spine fusion.

The following examples will serve to further illustrate the present invention without, at the same time, however, constituting any limitation thereof. On the contrary, it is to be clearly understood that resort may be had to, various embodiments, modifications and equivalents thereof which, after reading the description herein, may suggest themselves to those skilled in the art without departing from the spirit of the invention.

### EXAMPLE 1

### Preparation of a Composition Comprising a Solution of PDGF and a Biocompatible Matrix

A composition comprising a solution of PDGF and a biocompatible matrix of β-TCP was prepared according to the following procedure. The β-TCP comprised β-TCP particles having an average diameter ranging from about 1000 μm to about 2000 μm.

A solution comprising rhPDGF-BB was obtained. rhPDGF-BB is commercially available from Chiron Corporation at a stock concentration of 10 mg/ml (i.e., Lot # QA2217) in a sodium acetate buffer. The rhPDGF-BB is produced in a yeast expression system by Chiron Corporation and is derived from the same production facility as the rhPDGF-BB that is utilized in the products REGRANEX, (Johnson & Johnson) and GEM 21S (BioMimetic Therapeutics) which has been approved for human use by the United States Food and Drug Administration. This rhPDGF-BB is also approved for human use in the European Union and Canada. The rhPDGF-BB solution was diluted to 0.3 mg/ml in the acetate buffer. The rhPDGF-BB solution can be diluted to any desired concentration according to embodiments of the present invention, including 1.0 mg/ml.

A ratio of about 3 ml of rhPDGF-BB solution to about 1 g dry weight of the β-TCP biocompatible matrix was used to produce the composition. The rhPDGF-BB solution was expelled on the β-TCP particles of the biocompatible matrix with a syringe, and the resulting composition was blended and molded.

### EXAMPLE 2

### Preparation of a Composition Comprising a Solution of PDGF, a Biocompatible Matrix and a Biocompatible Binder

A composition comprising a solution of PDGF and a biocompatible matrix containing a biocompatible binder, collagen, was prepared according to the following procedure.

A pre-weighed block of biocompatible matrix comprising β-TCP and collagen was obtained. The β-TCP comprised β-TCP particles having an average diameter ranging from about 100 μm to about 300 μm. The p-TCP particles were formulated with approximately 20 weight percent soluble bovine collagen binder. A β-TCP/collagen matrix can be commercially obtained from Kensey Nash (Exton, Pa.).

A solution comprising rhPDGF-BB was obtained. rhPDGF-BB is commercially available from Chiron Corporation at a stock concentration of 10 mg/ml (i.e., Lot # QA2217) in a sodium acetate buffer. The rhPDGF-BB is produced in a yeast expression system by Chiron Corporation and is derived from the same production facility as the rhPDGF-BB that is utilized in the products REGRANEX, (Johnson & Johnson) and GEM 21S (BioMimetic Therapeutics) which has been approved for human use by the United States Food and Drug Administration. This rhPDGF-BB is also approved for human use in the European Union and Canada. The rhPDGF-BB solution was diluted to 0.3 mg/ml in the acetate buffer. The rhPDGF-BB solution can be diluted to any desired concentration according to embodiments of the present invention, including 1.0 mg/ml.

A ratio of about 3 ml of rhPDGF-BB solution to about 1 g dry weight of the β-TCP/collagen matrix was used to produce the composition. The rhPDGF-BB solution was expelled on the βTCP/collagen matrix with a syringe, and the resulting composition was blended and molded.

### EXAMPLE 3

### Preparation and Administration of Augment Bone Graft

Augment™ Bone Graft (rhPDGF-BB/β-TCP) is a completely synthetic bone graft substitute composed of recombinant human platelet-derived growth factor BB (0.3 mg/ml in 20 mM sodium acetate buffer) and beta-tricalcium phosphate granules. The beta-tricalcium phosphate particle size ranges from approximately 1000 to 2000 microns in diameter (purchased from Cam Bioceramics (Leiden, Netherlands)).

The components of Augment™ Bone Graft were provided in two sterile trays: The large tray contained a vial aseptically filled with rhPDGF-BB solution (3 ml, 0.3 mg/ml), a disposable syringe and disposable needle. The large tray was sterilized by ethylene oxide. The small tray contained a sealed cup filled with dry β-TCP granules. The small tray was sterilized by gamma radiation.

The composition was prepared as follows:

1) Using sterile technique, the cup (containing the β-TCP granules) and the vial (containing the rhPDGF-BB solution) was transferred to the sterile field.

2) The cup was opened and the β-TCP granules transferred to a sterile surgical bowl.

3) Using a syringe and needle, the contents of the vial were drawn up in entirety and all of the fluid transferred to the surgical bowl containing the β-TCP granules. If multiple kits were used (not to exceed 9 cc), the contents were combined.

4) The two components were gently stirred together for approximately 30 seconds using a spatula, curette or similar instrument.

5) The mixture was left undisturbed for 10 minutes before being implanted to ensure optimal saturation the β-TCP particles.

6) The product was implanted within one (1) hour after mixing the two components.

The composition is administered as follows:

At time of use, the two primary components are combined in entirety and mixed as described above, and applied to the surgical site.
The joint surfaces are debrided and decorticated to expose viable bone.
Where practical, surgical manipulations of the graft site are completed prior to implanting the graft material.
The surgical site is irrigated
Augment™ Bone Graft is manually packed into all subchondral voids and surface irregularities throughout the joint. Overfilling of the osseous defect(s) is avoided in order to achieve adequate fixation, closure and containment of the material.
The joint is reduced and rigid fixation is applied.
Any remaining Augment™ Bone Graft is packed around the perimeter of the joint.
All remaining rhPDGF-BB solution is applied to the surgical site to ensure the graft remains hydrated.
The periosteal and overlying soft tissue are carefully layered to enclose and contain the graft material. The graft site is not irrigated following implantation of Augment™ Bone Graft.

### EXAMPLE 4

### Preparation and Administration of Augment Injectable Bone Graft

Augment™ Injectable Bone Graft (rhPDGF-BB/β-TCP/Bovine Type I Collagen) is a synthetic bone graft substitute composed of recombinant human platelet-derived growth factor BB, beta-tricalcium phosphate granules and soluble bovine type I collagen. The beta-tricalcium phosphate particle size ranges from approximately 100 to 300 microns in diameter. Beta-tricalcium phosphate and collagen were purchased from Kensey Nash. The ratio of beta-tricalcium phosphate:collagen was 80:20 (w/w). Bovine Type I collagen component was added to enhance the handling characteristics of the product. The collagen component allows for the product to be formulated with 0.3 mg/ml rhPDGF-BB (in 20 mM Sodium Acetate buffer) solution to yield a flowable paste.

The components of Augment™ Injectable Bone Graft were provided in a “kit” consisting of two sterile containers: (1) The tray contained a vial aseptically filled with rhPDGF-BB solution (3 ml, 0.3 mg/ml). The tray was sterilized by ethylene oxide. (2) A double foil/clear pouch which contained 1 gram of β-TCP/Bovine Type 1 Collagen Matrix. The pouch was sterilized by gamma radiation.

The composition was prepared as follows:

1. Augment™ Injectable Bone Graft was prepared by completely saturating the β-TCP/collagen matrix with the rhPDGF-BB solution into a sterile surgical bowl under aseptic technique. If multiple kits were required (not to exceed 3 kits total), the contents were combined.

2. After completely saturating the β-TCP/collagen matrix, the mixture was left to sit for approximately 2 minutes. The mixture was then mixed with a non-glass spatula for 3 minutes until a smooth paste was formed. Properly mixed material had a uniform consistency without large chunks or pieces of solid material.

The composition is administered as follows:

At time of use, the two primary components are combined in entirety and mixed as described above, and applied to the surgical site. Following exposure of the bony defect, the bony void is adequately debrided and prepared according to standard bone grafting procedures.

1. The saturated matrix is carefully applied to the bone graft site. For more precise placement, Augment Injectable Bone Graft is packed into a sterile syringe using a cannula or large bore needle (not narrower than 16 gauge in size) and is injected/extruded into the target area(s).

2. In order to enhance the formation of new bone, Augment Injectable Bone Graft is placed in direct contact with well-vascularized bone. Cortical bone is perforated prior to placement of the Augment Injectable Bone Graft material.

3. The material is manually placed into the bone defect such that the graft material is in contact with the entire osseous surfaces to be fused.

4. Augment Injectable Bone Graft is also placed around the fusion site following fixation such that the growth factor may enhance periosteal bone formation.

5. Care is taken to ensure that Augment™ Injectable Bone Graft material is contained within the fusion space.

6. Once Augment Injectable Bone Graft is packed into the defect site, periosteal and overlying soft tissue are carefully layered to enclose and contain the graft material. This minimizes washout, subperiosteal resorption, exostosis, and ulceration at the surgical site. Care is taken not to irrigate the graft site following implantation of Augment™ Injectable Bone Graft.

7. Standard surgical techniques are employed to complete the procedure.

### EXAMPLE 5

### Preparation and Administration of Augment Injectable Bone Graft

Augment Injectable Bone Graft (rhPDGF-BB/Flowable β-TCP) is a synthetic bone graft substitute composed of recombinant human platelet-derived growth factor BB, beta-tricalcium phosphate granules and soluble bovine type I collagen. rhPDGF-BB is provided in a solution of 20 mM sodium acetate buffer at a concentration of 0.3 mg/mL. The beta-TCP particle size ranges from approximately 100 to 300 microns in diameter. A shredded Bovine Type I Collagen is added to enhance the handling characteristics of the product. Upon hydration with rhPDGF-BB solution, the collagen, in combination with the β-TCP, yields a flowable paste. Collagen and beta-TCP are purchased from Kensey Nash.

Augment Injectable Bone Graft is comprised up two primary sterile components: (1) A tray containing an aseptically filled vial with rhPDGF-BB solution (3 ml, 0.3 mg/ml). The tray is sterilized by ethylene oxide. (2) A foil/clear pouch containing 1 gram of β-TCP/Bovine Type I Collagen Matrix (80%/20% w/w) in a 10 cc polypropylene syringe, an empty polypropylene syringe, one 18 gauge blunt tip needle, one 14 gauge blunt tip needle and female/female luer connector. The pouch is sterilized by gamma radiation.

The composition is prepared and administered as follows:

At time of use, the two primary components are combined in entirety, mixed and applied to the surgical site.

Following exposure of the surgical site, the joint(s) are adequately debrided and prepared according to standard surgical technique. All remaining cartilage is removed and the opposing bony surfaces are adequately prepared to optimize apposition of healthy, vascularized bone. This is done by feathering and/or perforating the remaining subchondral plate with standard use of curettes, burrs, drill bits or osteotomes as a means of maximizing the surface area of exposed bleeding bone prior to insertion of the graft.

Augment Injectable Bone Graft is then prepared by completely saturating the β-TCP/collagen matrix with the rhPDGF-BB solution, as shown in the following diagram, and is administered as follows:

1. The contents of the vial containing the rhPDGF-BB solution are completely withdrawn using the empty syringe and 18 gauge needle. After all of the fluid is extracted from the vial, the needle is removed and any air remaining in the syringe is displaced.

2. The cap from the syringe containing the β-TCP/collagen matrixis is removed. The plunger is pulled to the 10 ml mark and the syringe is tapped to loosen the matrix. The plunger is returned to the 8 ml mark.

3. The syringe containing the rhPDGF-BB solution is connected with the syringe containing the matrix using the female-to-female luer-lock connector.

4. The rhPDGF-BB solution is transferred into the syringe containing the matrix. After transferring all of the rhPDGF-BB solution, the plunger on the syringe containing the hydrated matrix is pulled to the 10 ml mark.

5. The plunger of the syringe containing the hydrated matrix is released. The syringes are allowed to sit undisturbed for a minimum of 90 seconds.

6. After hydrating the matrix, the contents are transferred back and forth between the two syringes for no less than (20) twenty cycles. A cycle is defined as passing the matrix to the empty syringe and back. Upon completion, the matrix forms a homogenous paste.

7. All of the paste is transferred to one of the syringes, and any pressure built up during the mixing process is relieved by gently pulling the plunger containing the matrix.

8. The empty syringe and female-to-female luer-lock connector from the syringe that contains the paste are disconnected. Any air remaining in the syringe is displaced and the 14 gauge needle is connected. The hydrated matrix is dispensed into the void. Where necessary, an initial force is applied to get the paste to flow through the 14 gauge needle. However, once the paste starts to flow the force required to maintain a flow is reduced.

9. The hydrated matrix is carefully applied to the surgical site (i.e., the subchondral voids, and surface irregularities visualized throughout the entire joint) immediately after joint reduction and screw fixation of the fusion site. Any remaining (unused) Augment Injectable Bone Graft is packed around the external perimeter of the fusion construct.

10. In order to enhance the formation of new bone, Augment Injectable Bone Graft is placed in direct contact with well-vascularized bone. Cortical bone is perforated prior to placement of the Augment Injectable Bone Graft material.

11. Once Augment Injectable Bone Graft is packed into the defect site, periosteal and overlying soft tissue are carefully layered to enclose and contain the graft material. This minimizes washout, subperiosteal resorption, exostosis, and ulceration at the surgical site. Care is taken not to irrigate the graft site following implantation of Augment Injectable Bone Graft.

12. Standard surgical techniques are employed to complete the procedure.

13. Any remaining graft material is discarded.

### EXAMPLE 6

Determination of Interbody Lumbar Spine Fusion in Sheep Following Treatment With Augment™ Bone Graft and Augment™ Injectable Bone Graft

### Purpose

The purpose of this study was to determine the ability of different matrices containing rhPDGF-BB (β-TCP, β-TCP/Collagen) compared with autograft to promote interbody fusion (bony bridging) of the L2/L3 and L4/L5 vertebral bodies in an ovine spinal fusion model.

### Test Facility

The in vivo part of the study including surgeries, in-life follow-up, radiographic imaging and necropsies were performed at the Small Ruminant Comparative Orthopedic Laboratory of the Department of Clinical Sciences at Colorado State University in Fort Collins, Co. MicroCT imaging and histological processing and assessment were conducted in the R&D Laboratory at the BioMimetic Therapeutics, Inc. Franklin, Tenn. facility.

### Study Design

Twenty-two (22) sheep were scheduled to receive an un-instrumented, double-level, lateral interbody lumbar spinal fusion procedure using a polyetheretherketone (PEEK) spacer as a vertebral spacer.

The PEEK vertebral spacer was packed with one of the following matrices: Group 1—Empty; Group 2—Iliac crest autograft; Group 3—Augment Bone Graft (ABG; (3-TCP+0.3 mg/mL rhPDGF-BB); Group 4—Augment Injectable Bone Graft (AIBG; (3-TCP/Collagen+0.3 mg/mL rhPDGF-BB). Groups 3 and 4 were the test articles being evaluated; and Group 2 was the positive control group and Group 1 was the negative control group.

The same treatment was used at both the L2/L3 and L4/L5 levels within each sheep, in order to avoid possible diffusion between levels, or a systemic effect of the biologic material. There were five animals corresponding to 10 fusion levels evaluated in Groups 2-4, and seven animals corresponding to 14 fusion levels evaluated in Group 1. Lateral and anteroposterior view radiographs of the lumbar spine from LI to L6 were taken at 0, 12 and 24 weeks after surgery. All animals were sacrificed at 24 weeks after surgery and the fusion sites removed en bloc. Fusion was assessed by microCT and histolologic analyses.

### Species

Twenty-two (22) mature, female Rambouillet x Columbia sheep were used for this study. All sheep were acquired from a single commercial source and had a minimum 28 day acclimation period prior to participation in the study. Sheep were ear tagged for unique individual animal identification. Physical examinations were performed to identify and replace any unhealthy animals. All animals were dewormed and housed in the large animal research barn around the time of surgery and then in a pasture. All animals were fed a diet of grass/alfalfa hay mix throughout the acclimatization and study period. Daily animal care was provided by SRCOL staff members and the CSU Laboratory Animal Resources group.

All procedures involving the use of live animals were approved by the Colorado State University IACUC.

### Sample Size

A total of 22 animals underwent spinal fusion using a polyetheretherketone (PEEK) spacer as a vertebral spacer. The animals received the PEEK spacer packed with one of the following, with the same treatment at both the L2/L3 and L4/L5 levels: Group 1—Empty (n=7 animals; 14 fusion sites); Group 2—Autograft (n=5 animals; 10 fusion sites); Group 3—ABG (n=5 animals; 10 fusion sites); Group 4—AIBG (n=5 animals; 10 fusion sites).

### Surgical Method

Surgeries were performed at the research facility site. Representatives from the study sponsor were present for the surgical procedures. Operative record data forms were completed at the time of surgery and included surgeons, treatment allocation group, time from incision to closure, as well as any unusual findings/events at the time of surgery.

On the day of surgery, acepromazine maleate (0.05 mg/kg 1M) and Buprenorphine (0.005-0.01 mg/kg 1M) were administered prior to anesthestic induction. An IV injection consisting of Diazepam (0.22 mg/kg) and Ketamine (10 mg/kg) was given for induction of general anesthesia. A cuffed endotracheal tube was placed and general anesthesia was maintained with halothane (1.5% to 3.0%) in 100% oxygen (2 L/min) through a rebreathing system. The animal was placed on a ventilator to assist respiration

With the animal in right lateral recumbency, the wool was removed from the left lateral lumbar area. The skin over the left lateral lumbar area and iliac crest area (autograft group only) were prepared for aseptic surgery using alternating scrubs of povidone-iodine (Betadine) and alcohol. The area was then be draped for aseptic surgery and a lateral retroperitoneal approach to the disc spaces of L2/L3 and L4/L5 was be made. First, the disc space of L4/L5 was identified and an anulotomy performed. Using a Midas-Rex burr, the endplate was prepared to a size to accept the Vertebral Spacer-CR spacer.

Before insertion of the vertebral spacer, a vertebral spreader was used to open the disc space. The spacer, plus its contents (0.4 mL) were pressed into place. The same procedure was performed at L2/L3, with the same test article as was used at the L4/L5 level, based on the experimental design. Routine closure of external muscular fascia (0 Polysorb absorbable suture, subcutaneous tissue (2/0 Polysorb) and skin (2/0 monofilament non-absorbable suture, Ford interlocking pattern) was performed. Perioperative antibiotics (Cephazolin sodium) were administered.

### Preparation of Materials

Iliac Crest Autograft Harvesting. The dorsal and dorsolateral lumbar and iliac crest areas were prepared for aseptic surgery with multiple scrubs of povidone-iodine alternated with isopropyl alcohol. The area was draped and a 3-cm incision made over the iliac crests. Following partial reflection of the gluteal muscles, a curette was used to remove approximately 1 cc of autologous cancellous bone, later to be inserted in the Vertebral Spacer-CR spacer at L2/L3 and L4/L5 of the positive control sheep. Intralesional morphine sulfate (1.5 mL (22.5 mg total)) was administered prior to closure of the iliac crest incisions. The incisions over the iliac crest were closed routinely using 2/0 Polysorb for the subcutaneous tissues and stainless steel staples for the skin.

ABG. Prior to implantation, the ABG graft material was prepared according to Example 3. The hydrated ABG was allowed to sit at room temperature for 5-15 minutes and then transferred to a syringe with the end removed. The syringe was used to dispense an accurate volume to the interior of the PEEK spacer (0.4 mL).

AIBG. Prior to implantation, the AIBG graft material was prepared according to Example 4. The hydrated AIBG was allowed to sit at room temperature for 5-15 minutes and then transferred to a syringe with the end removed. The syringe was used to distribute an accurate volume to the interior of the PEEK spacer (0.4 mL).

### Aftercare

Immediately after surgery, the sheep was transferred from the operating table to radiology for postoperative radiographs of the lumbar spine to verify appropriate PEEK spacer implant placement and provide baseline radiographic imaging for fusion assessment. They were then taken to an aluminum stock trailer where they were positioned in sternal recumbency. At the end of the day, all operated sheep were moved to the research barn at the Veterinary Medical Center. All sheep made uneventful recoveries from surgery and anesthesia. The sheep were housed indoors for the first two weeks of the study to monitor healing of the incision sites. Postoperative analgesia was provided with fentanyl patches and 3 days of oral phenylbutazone. Animals were allowed to ambulate normally for the 24 weeks of the study period.

### In-Life Observations and Imaging

Clinical Observations. All sheep made uneventful recoveries from surgery and anesthesia. Animals were observed twice daily throughout the post-surgical study period for general attitude, appetite, appearance of the surgical site, neurological signs and respiratory stress. Daily observations and any adverse events were recorded in an Excel spreadsheet by the SRCOL staff. All animals survived the 24 week study period and there were no unscheduled animal deaths during this study

Radiographs. Immediately post-operatively, lateral and anterioposterior radiographs of the lumbar spine were taken to include the two surgical sites (L2/L3 and L4/L4) for baseline readings and to assess implant placement. Radiographs were also obtained at 12 weeks (in-vivo) and 24 weeks (explanted spine) after surgery. After imaging all animals were returned to their housing unit.

### Necropsy and Specimen Collection and Handling

All animals were euthanized by intravenous overdose of pentobarbitone sodium, in accordance with the AVMA 2007 guidelines, twenty-four (24) weeks after surgery. The lumbar spines were explanted following euthanasia and the soft tissues removed. Each spinal unit was radiographed as described above.

### MicroCT Analysis

MicroCT scanning and analysis was performed on a μCT 80 system (SCANCO USA, Southeastern, Pa.) using the manufacturer's analysis software. Endpoints for microCT analysis include assessment of bony bridging throughout the central cavity of the vertebral spacer and the bone volume/total volume (BV/TV) of the central cavity.

Additionally, differential density analyses were performed in groups 2 (Autograft), 3 (ABG), and 4 (AIBG) to ascertain the presence of residual β-TCP in the repair tissue.

### Histologic Analysis

Harvested and trimmed specimens were placed in 10% neutral buffered formalin (NBF) overnight, changed with fresh 10% NBF, and then shipped overnight to BioMimetic Therapeutics (BMTI) to complete fixation and in preparation for undecalcified histology.

Upon arrival at BMTI, the specimens were accessioned, trimmed again when necessary, and changed into fresh 10% NBF where they remained for approximately one week under vacuum. The specimens were dehydrated in several changes of graded EtOH solutions and cleared with xylenes and methyl methacrylate (MMA). Next, the specimens were infiltrated under vacuum, using three solutions (Infiltration Solutions I, II, and III) containing MMA and dibutyl phthalate (DBP). Upon completion, the specimens were embedded in a fresh solution of MMA+DBP and Perkadox-16 and allowed to polymerize.

Representative histological sections throughout the central region of the vertebral spacer (primary endpoint) were obtained from each level using the EXAKT Cutting/Grinding system (EXAKT Technologies, Inc., Oklahoma City, Ok.). Additional sections were taken from the area surrounding the vertebral spacer (secondary endpoint). All sections were then “ground” to an appropriate thickness and stained using a metachromatic stain (Sanderson's Rapid Bone Stain) alone and/or in combination with a counterstain (Van Gieson picrofuschin) to yield a traditional trichrome stain used in the assessment of bone morphology.

Following processing, sectioning, and staining, individually labeled sections (with unique identifier numbers) were graded based on the following scoring method (Toth, J., et al., Evaluation of 70/30 poly (L-lactide-co-D,L-lactide) for use as a resorbable interbody fusion cage. Journal of Neurosurgery: Spine, 2002. 97(4 Suppl): p.423-432; Sandhu, H. S., et al., Histologic evaluation of the efficacy of rhBMP-2 compared with autograft bone in sheep spinal anterior interbody fusion. Spine, 2002. 27(6): p. 567575; Toth, J. M., Wang, M., Estes, B. T., Scifert, J. L., Seim, H. B., Turner, A S., Polyetheretherketone as a biomaterial for spinal applications. Biomaterials, 2006. 27(3 (Special Issue)): p. 324-334.):

Total fusion: more than 50% of slides showed continuous bony bridging;

Partial fusion: less than 50% of slides showed continuous bony bridging;

Non fusion: no continuous bony bridging.

### Statistical Methods

Comparison of treatment groups was carried out using ANOVA on ranks with post-hoc Dunn's test for non parametric data (microCT and histology fusion scores) and One-way ANOVA with Holm-Sidak post hoc test for parametric data (bone volume over total volume and mineral density) to determine the differences between groups.

### Results

### MicroCT

Statistical analysis revealed differences among the groups (ANOVA on Ranks; p=0.021) with ABG having significantly higher fusion rate than the Empty control (post-hoc Dunn's test). No significant differences were detected among the fusion scores on Autograft, ABG or AIBG.

All the treatment groups had at least one specimen with a successful fusion (score of 2.00). The ABG- and AIBG-treated groups both had 6 specimens which scored as completely fused (Table 2) while the Empty and Autograft groups had only two and three respectively.

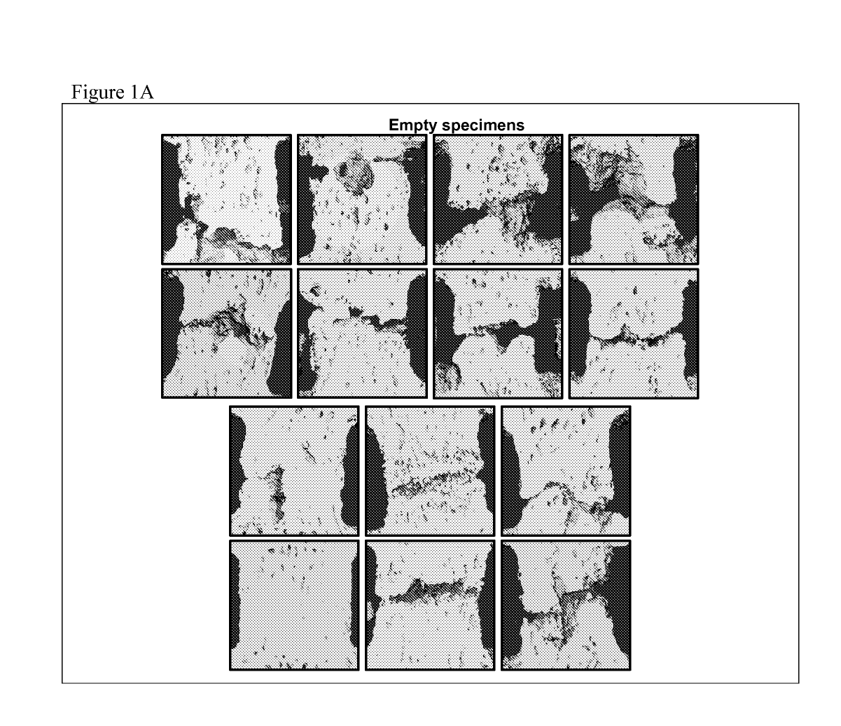

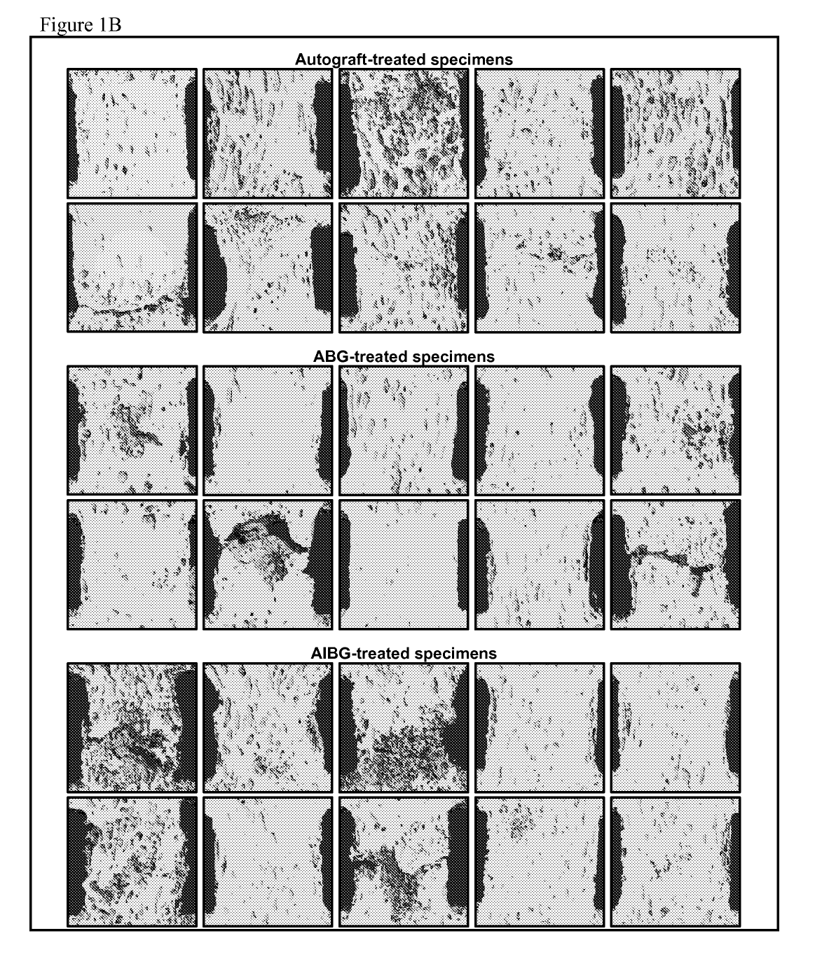

A summary of the microCT fusion scores for each treatment group is shown in Table 1; individual microCT fusion scores are shown in Table 2. Representative microCT images from each specimen are shown in FIGS. 1A and 1B.

TABLE 1
MicroCT Fusion scores for each treatment group.

| Group | Mean | Std. Dev. | Median | Max | Min |
|---|---|---|---|---|---|
| Empty | 0.72 | 0.62 | 0.61 | 2.00 | 0.00 |
| Autograft | 1.63 | 0.48 | 1.81 | 2.00 | 0.67 |
| ABG* | 1.58 | 0.78 | 2.00 | 2.00 | 0.00 |
| AIBG | 1.44 | 0.74 | 2.00 | 2.00 | 0.22 |

*Different from Empty; p = 0.021

TABLE 2
MicroCT fusion scores for each individual specimen.

| Empty | | Autograft | | ABG | | AIBG | |
|---|---|---|---|---|---|---|---|
| ID | Score | ID | Score | ID | Score | ID | Score |
| 02A | 0.00 | 28A | 1.78 | 48A | 1.72 | 54A | 0.22 |
| 02B | 1.00 | 28B | 0.89 | 48B | 2.00 | 54B | 0.83 |
| 08A | 0.89 | 34A | 2.00 | 49A | 2.00 | 55A | 2.00 |
| 08B | 0.61 | 34B | 1.67 | 49B | 0.00 | 55B | 2.00 |
| 15A | 0.61 | 41A | 0.67 | 50A | 2.00 | 56A | 0.94 |
| 15B | 0.11 | 41B | 1.94 | 50B | 2.00 | 56B | 0.44 |
| 18A | 0.50 | 47A | 1.83 | 51A | 2.00 | 57A | 2.00 |
| 18B | 0.39 | 47B | 1.50 | 51B | 2.00 | 57B | 2.00 |
| 22A | 2.00 | 53A | 2.00 | 52A | 1.89 | 58A | 2.00 |
| 22B | 2.00 | 53B | 2.00 | 52B | 0.22 | 58B | 2.00 |
| 23A | 0.89 | | | | | | |
| 23B | 0.61 | | | | | | |
| 25A | 0.39 | | | | | | |
| 25B | 0.06 | | | | | | |

Analysis of the bone volume over total volume (BV/TV; %) within the PEEK spacer revealed no differences among the treatment groups (One-way ANOVA, p=0.308). A summary of the values for each treatment group is shown in Table 3, whereas individual BV/TV values are shown in Table 4.

TABLE 3
Bone volume over total volume (%) for each treatment group.

| Group | Mean | Std. Dev. |
|---|---|---|
| Empty | 64.46% | 11.69% |
| Autograft | 67.22% | 14.77% |
| ABG | 75.82% | 15.39% |
| AIBG | 63.59% | 22.68% |

TABLE 4
Bone volume over total volume (%) for each individual specimen.

| EMPTY | | AUTOGRAFT | | ABG | | AIBG | |
|---|---|---|---|---|---|---|---|
| ID | BV/TV | ID | BV/TV | ID | BV/TV | ID | BV/TV |
| 02A | 53.60% | 28A | 82.14% | 48A | 68.33% | 54A | 27.53% |
| 028 | 69.42% | 288 | 79.18% | 488 | 76.94% | 548 | 39.69% |
| 08A | 64.53% | 34A | 63.36% | 49A | 77.41% | 55A | 60.92% |
| 088 | 64.14% | 348 | 70.81% | 498 | 47.70% | 558 | 83.14% |
| 15A | 56.97% | 41A | 35.87% | 50A | 77.15% | 56A | 42.26% |
| 158 | 60.24% | 418 | 65.14% | 508 | 95.22% | 568 | 47.88% |
| 18A | 52.34% | 47A | 73.74% | 51A | 90.59% | 57A | 84.98% |
| 188 | 69.05% | 478 | 76.83% | 518 | 93.12% | 578 | 87.84% |
| 22A | 80.48% | 53A | 48.33% | 52A | 75.59% | 58A | 84.59% |
| 228 | 93.96% | 538 | 76.80% | 528 | 56.18% | 588 | 77.08% |
| 23A | 58.91% | | | | | | |
| 23B | 64.07% | | | | | | |
| 25A | 56.43% | | | | | | |
| 25B | 49.23% | | | | | | |

Analysis of the density of the bone within the spacer revealed differences among the groups (One-way ANOVA, p<0.001). Density in the ABG group was higher than in the other groups (post-hoc Holm-Sidak test); AIBG and Autograft had lower density than Empty and were no different from each other. Individual bone density values (mg HA/cm³) are shown in Table 5; a summary of the values for each group is shown in Table 6.

TABLE 5
Bone density values (mg HA/cm3) for each individual specimen.

| Empty | | Autograft | | ABG | | AIBG | |
|---|---|---|---|---|---|---|---|
| ID | Density | ID | Density | ID | Density | ID | Density |
| 02A | 637.77 | 28A | 621.59 | 48A | 647.25 | 54A | 626.59 |
| 028 | 648.86 | 288 | 646.98 | 488 | 671.65 | 548 | 632.76 |
| 08A | 645.28 | 34A | 628.83 | 49A | 670.67 | 55A | 613.63 |
| 088 | 649.03 | 348 | 672.10 | 498 | 712.87 | 558 | 662.62 |
| 15A | 686.85 | 41A | 591.72 | 50A | 712.07 | 56A | 624.98 |
| 158 | 663.97 | 418 | 604.24 | 508 | 701.96 | 568 | 624.72 |
| 18A | 634.10 | 47A | 619.71 | 51A | 680.15 | 57A | 609.43 |
| 188 | 652.43 | 478 | 638.57 | 518 | 675.05 | 578 | 629.01 |
| 22A | 657.35 | 53A | 617.83 | 52A | 684.98 | 58A | 636.52 |
| 22B | 671.03 | 538 | 614.70 | 528 | 649.75 | 588 | 595.84 |
| 23A | 269.63 | | | | | | |
| 23B | 655.11 | | | | | | |
| 25A | 696.69 | | | | | | |
| 25B | 678.90 | | | | | | |

TABLE 6
Bone density values (mg HA/cm3) for each treatment group.

| Group | Mean | Std. Dev. |
|---|---|---|
| Empty# | 657.64 | 19.85 |
| Autograft | 625.63 | 22.69 |
| ABG*# | 680.64 | 23.05 |
| AIBG | 625.61 | 17.78 |

*Different from Empty; p < 0.001
#Different from AIBG and Autograft; p < 0.001

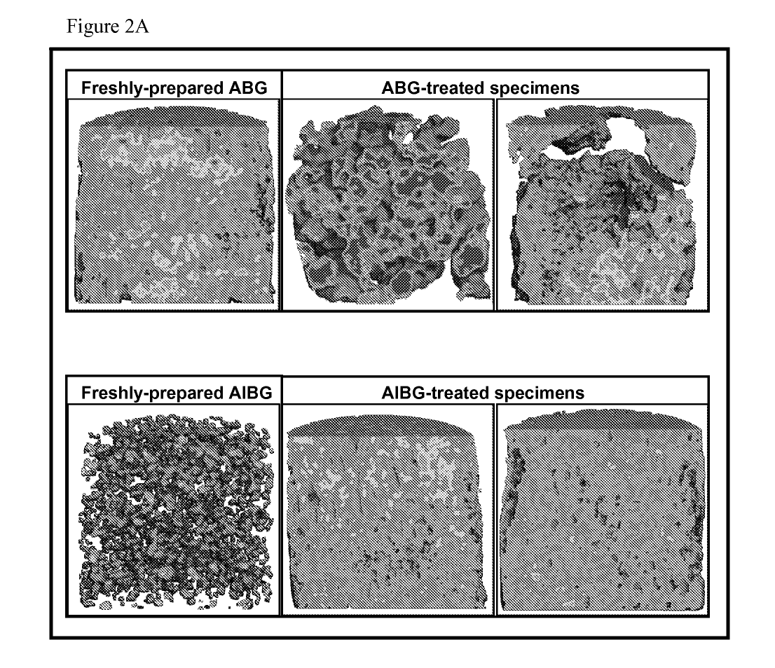

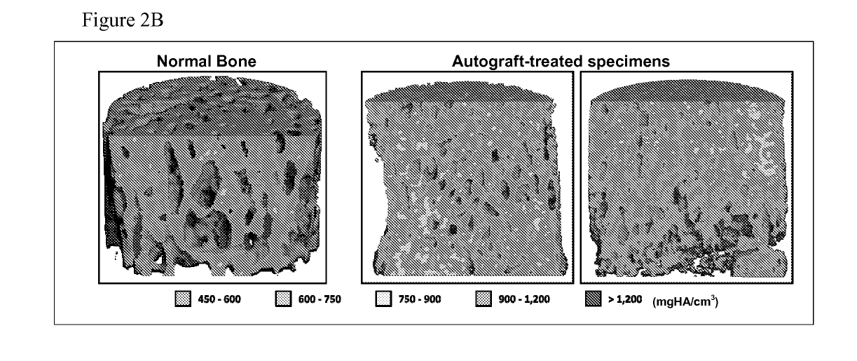

Detailed analysis of the mineral density of the bone within the PEEK spacer (Table 7 and FIGS. 2A and 2B) revealed that ABG-treated specimens exhibited areas with high mineral density (>900 mg HA/cm³) that likely correspond to residual β-TCP. These areas were not as conspicuous in AIBG-treated specimens and were not present in Autograft-treated or Empty specimens. The material density of the ABG-treated specimens is the one that most closely resembles that of normal bone. Table 7 shows a comparison of density in autograft, ABG, and AIBG treatment groups, as well as a freshly prepared ABG and AIBG.

TABLE 7
Bone density (mg HA/cm3) distribution for each treatment group

| Group | 450-600 | 600-750 | 750-900 | 900-1,200 | >1,200 |
|---|---|---|---|---|---|
| Autograft | 61% | 37% | 2% | 0% | 0% |
| ABG | 39% | 47% | 10% | 4% | 0% |
| AIBG | 64% | 34% | 2% | 0% | 0% |
| Normal bone | 47% | 50% | 10% | 4% | 0% |
| Freshly-prepared | 15% | 14% | 14% | 28% | 28% |
| ABG | | | | | |
| Freshly-prepared | 47% | 26% | 10% | 4% | 0% |
| AIBG | | | | | |

### Histology

Statistical analysis revealed differences among the groups (ANOVA on Ranks; p=0.008) with the fusion score of the ABG-treated group being significantly higher than that of the Empty control (post-hoc Dunn's test).

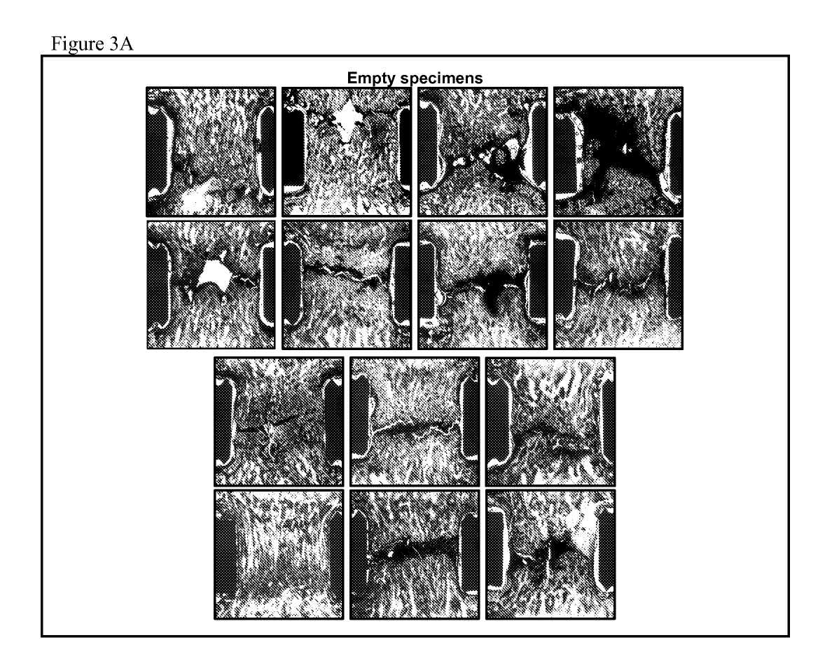

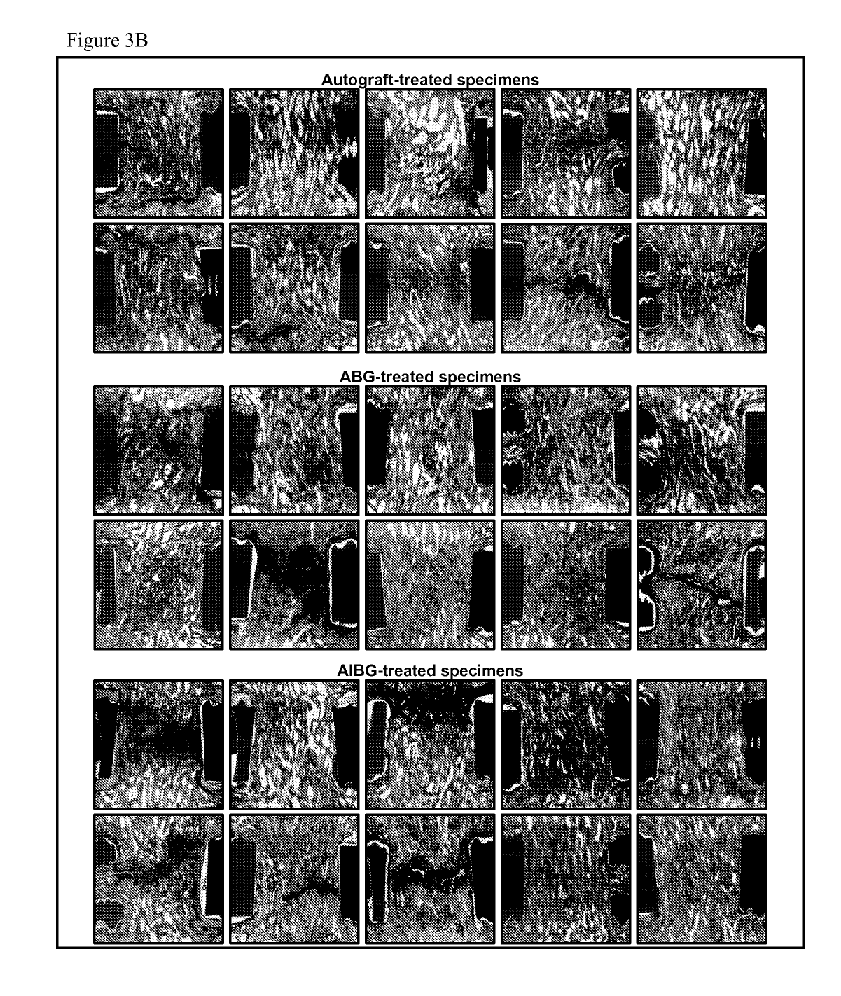

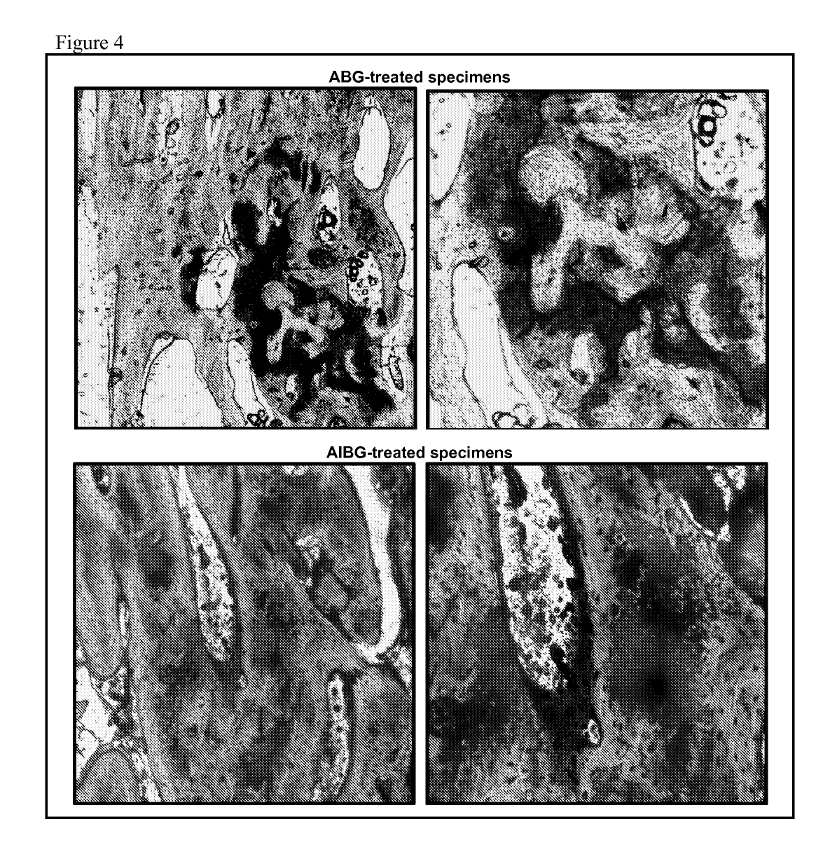

All the treatment groups had at least one specimen with a successful fusion (score of 2.00). The ABG-treated group had 7 specimens scored as completely fused (Table 9); the A1BG-treated and Autograft-treated groups had 5 of these specimens and the Empty group had only one in 14 specimens; the Empty group was also the only group with specimens scored as zero. Representative histological images from each treatment group are shown in FIGS. 3A and 3B. Residual β-TCP particles were visible in ABG- and AIBG-treated groups. These particles were not preferentially located in a specific area of the repair tissue but they appeared to be randomly located. The particles were surrounded by bone without any indication of fibrous encapsulation (FIG. 4). In some cases, the β-TCP particles were found in the area of failed fusion. This was the case in two of the specimens in the ABG-treated group in which the particles found in this area appeared to be of a very large size. Some of the areas that had not fused in AIBG-treated specimens presented cartilaginous tissue; in one of them this tissue was found around β-TCP particles.

A summary of the histology fusion scores for each group is shown in Table 8; individual histology fusion scores are shown in Table 9.

TABLE 8
Histology fusion scores for each treatment group.

| Group | Mean | Std. Dev. | Median | Max | Min |
|---|---|---|---|---|---|
| Empty | 0.61 | 0.51 | 0.58 | 2.00 | 0.00 |
| Autograft | 1.45 | 0.64 | 1.67 | 2.00 | 0.33 |
| ABG* | 1.62 | 0.73 | 2.00 | 2.00 | 0.17 |
| AIBG | 1.43 | 0.70 | 1.92 | 2.00 | 0.50 |

TABLE 9
Histology fusion scores for each individual specimen.
Mean of the average scores of 2 sections each
evaluated by 3 independent observers.

| Empty | | Autograft | | ABG | | AIGB | |
|---|---|---|---|---|---|---|---|
| ID | Score | ID | Score | ID | Score | ID | Score |
| 02A | 0.17 | 28A | 1.17 | 48A | 1.67 | 54A | 0.67 |
| 02B | 0.67 | 28B | 0.33 | 48B | 2.00 | 54B | 0.67 |
| 08A | 1.00 | 34A | 2.00 | 49A | 2.00 | 55A | 2.00 |
| 08B | 0.33 | 34B | 1.33 | 49B | 0.17 | 55B | 1.83 |
| 15A | 0.00 | 41A | 1.00 | 50A | 2.00 | 56A | 0.50 |
| 15B | 0.00 | 41B | 2.00 | 50B | 2.00 | 56B | 0.67 |
| 18A | 0.33 | 47A | 2.00 | 51A | 2.00 | 57A | 2.00 |
| 18B | 0.67 | 47B | 0.67 | 51B | 2.00 | 57B | 2.00 |
| 22A | 1.00 | 53A | 2.00 | 52A | 2.00 | 58A | 2.00 |
| 22B | 2.00 | 53B | 2.00 | 52B | 0.33 | 58B | 2.00 |
| 23A | 0.67 | | | | | | |
| 23B | 0.67 | | | | | | |
| 25A | 0.50 | | | | | | |
| 25B | 0.50 | | | | | | |

### Conclusions

The ABG-treated specimens had the highest fusion scores of all groups evaluated. ABG significantly promoted interbody spine fusion compared to empty PEEK spacers.

### REFERENCES

Sandhu, H. S., et al., Distractive Properties of a Threaded Interbody Fusion Device: An In Vivo Model. Spine, 1996.21(10): p. 1201-1210.
Sandhu, H. S., et al., Animal models of spinal instability and spinal fusion, in Animal Models in Orthopaedic Research, Y. H. An and R. J. Friedman, Editors. 1999, CRC Press: Boca Raton.

Toth, J. M., et al., Direct current electrical stimulation increases the fusion rate of spinal fusion cages. Spine, 2000. 25(20): p. 2580-2587.

Wilke, H., A Kettler, and L. Claes, Are sheep spines a valid biomechanical model for huma spines? Spine, 1997.22(20): p. 2365-2374.

Wilke, H.-J., et al., Anatomy of the sheep spine and its comparison to the huma spine. The Anatomical Record, 1997.247(4): p. 542-555.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

1. A method of promoting bone fusion in a spine fusion procedure, comprising administering to a site of desired spine fusion a composition comprising:
a biocompatible matrix and a solution comprising platelet derived growth factor (PDGF), wherein the solution is incorporated in the biocompatible matrix, wherein the biocompatible matrix comprises a bone scaffolding material, and wherein the bone scaffolding material comprises a porous calcium phosphate or allograft.

2. The method according to claim 1, wherein the bone scaffolding material comprises calcium phosphate.

3. The method according to claim 1, wherein the calcium phosphate comprises β-tricalcium phosphate.

4. The method according to claim 1, wherein the bone scaffolding material comprises allograft.

5. The method according to claim 1, wherein the PDGF is present in the solution at a concentration from about 0.01 mg/ml to about 10.0 mg/ml.

6. The method according to claim 1, wherein the PDGF is present in the solution at a concentration from about 0.05 mg/ml to about 5.0 mg/ml.

7. The method according to claim 1, wherein the PDGF is present in the solution at a concentration from about 0.1 mg/ml to about 1.0 mg/ml.

8. The method according to claim 1, wherein the PDGF is present in the solution at a concentration from about 0.2 mg/ml to about 0.4 mg/ml.

9. The method according to claim 1, wherein the PDGF is present in the solution at a concentration of about 0.3 mg/ml.

10. The method according to claim 1, wherein the PDGF comprises PDGF-AA, PDGF-BB, PDGF-AB, PDGF-CC, PDGF-DD, or a mixture or a derivative thereof.

11. The method according to claim 1, wherein the PDGF comprises PDGF-BB.

12. (canceled)

13. The method according to claim 1, wherein the PDGF-BB comprises at least 65% intact PDGF-BB.

14. The method according to claim 1, wherein the PDGF-BB is recombinant human (rh)PDGF-BB.

15-17. (canceled)

18. The method according to claim 1, wherein the bone scaffolding material comprises particles in a range of about 50 microns to about 5000 microns in size.

19. (canceled)

20. The method according to claim 1, wherein the bone scaffolding material comprises particles in a range of about 100 microns to about 5000 microns in size.

21. (canceled)

22. The method according to claim 1, wherein the bone scaffolding material comprises particles in a range of about 100 microns to about 300 microns in size.

23. (canceled)

24. The method according to claim 1, wherein the bone scaffolding material comprises particles in a range of about 1000 microns to about 2000 microns in size.

25. (canceled)

26. The method according to claim 1, wherein the bone scaffolding material comprises particles in a range of about 250 microns to about 1000 microns in size.

27. (canceled)

28. The method according to claim 1, wherein the bone scaffolding material comprises particles in a range of about 1000 microns to about 3000 microns in size.

29. (canceled)

30. The method according to claim 1, wherein the bone scaffolding material comprises porosity greater than about 25%.

31-34. (canceled)

35. The method according to claim 1, wherein the bone scaffolding material comprises macroporosity.

36. The method according to claim 1, wherein the bone scaffolding material has a porosity that facilitates cell migration into the matrix.

37. The method according to claim 1, wherein the bone scaffolding material comprises interconnected pores.

38. The method according to claim 1, wherein the bone scaffolding material is resorbable.

39. (canceled)

40. The method according to claim 1, wherein the solution is absorbed or adsorbed to the bone scaffolding material.

41. The method according to claim 1, wherein the bone scaffolding material is capable of absorbing an amount of the solution that is equal to at least about 25% of the bone scaffolding's own weight.

42-45. (canceled)

46. The method according to claim 1, wherein the biocompatible matrix further comprises a biocompatible binder.

47. The method according to claim 46, wherein the biocompatible binder comprises collagen.

48. The method according to claim 47, wherein bone scaffolding material and collagen are present in a ratio of about 80:20.

49-52. (canceled)

53. The method according to claim 1, wherein the method comprises:
performing a spine fusion procedure on a patient;
applying the composition to the site of desired spine fusion; and, permitting bone fusion to occur at the site.

54. The method of claim 1, wherein the spine fusion procedure is an interbody fusion procedure.

55. The method of claim 1, wherein the spine fusion procedure is a lumbar fusion procedure.

56. The method of claim 1, wherein the spine fusion procedure comprises accelerating bony union.

57-114. (canceled)
